(19) United States
(12) Patent Application Publication
(10) Pub. No.: US 2026/0062692 A1
(43) Pub. Date: Mar. 5, 2026

(54) COMPOSITIONS AND METHODS FOR THE IMMOBILIZATION OF ENZYMES USING CROSSLINKED BIOMOLECULAR CONDENSATES

(71) Applicant: RUTGERS, THE STATE UNIVERSITY OF NEW JERSEY, New Brunswick, NJ (US)

(72) Inventors: Benjamin Schuster, Highland Park, NJ (US); Augene Park, New Brunswick, NJ (US)

(73) Assignee: RUTGERS, THE STATE UNIVERSITY OF NEW JERSEY, New Brunswick, NJ (US)

(21) Appl. No.: 19/314,472

(22) Filed: Aug. 29, 2025

Related U.S. Application Data
(60) Provisional application No. 63/688,488, filed on Aug. 29, 2024.

(51) Int. Cl. C12N 11/18; C12N 9/04; C12N 9/06; C12N 9/10; C12N 9/14

(52) U.S. Cl. CPC C12N 11/18; C12N 9/0006; C12N 9/0016; C12N 9/1096; C12N 9/14; C12Y 101/01001; C12Y 104/01001; C12Y 206/01048; C12Y 306/04013

(57) ABSTRACT

Compositions and methods for the preparation of immobilized enzymes for use in biocatalytic processes are provided. More specifically, the immobilized enzyme systems are covalently cross-linked biomolecular condensates that stabilize the enzymes without impeding their effectiveness as biocatalyst.

### CROSS-REFERENCE TO RELATED APPLICATION

This application claims priority to U.S. Provisional Application No. 63/688,488, filed Aug. 29, 2024, which is incorporated by reference as though set forth in full.

### GOVERNMENT INTEREST STATEMENT

This invention was made with government support under grant number NSF 2238914 awarded by the National Science Foundation. The government has certain rights in the invention.

### FIELD OF THE INVENTION

This invention relates to the field of enzyme immobilization. More specifically, compositions and methods are provided which immobilize enzymes by covalently crosslinking biomolecular condensates for industrial and research use.

### BACKGROUND OF THE INVENTION

Several publications and patent documents are cited throughout the specification in order to describe the state of the art to which this invention pertains. Each of these citations is incorporated herein by reference as though set forth in full.

Catalysis serves as the cornerstone of modern industrial processes and plays a pivotal role in society, encompassing applications from the generation of electricity to pharmaceuticals manufacturing. Many industrial processes heavily rely on heterogeneous catalysts, which can exert detrimental effects on the environment. With recent advancements in biotechnology, there has been a resurgence of interest in engineering enzymes for utilization in biocatalytic processes. Nevertheless, free-floating enzymes can be difficult to use in biocatalysis because of 1) their inherent instability in extreme operational conditions, 2) the need for complicated bioseparation techniques at the conclusion of the reaction, and 3) their limited reusability. Consequently, enzymes are often immobilized for easier handling. However, existing immobilization techniques can introduce new issues that impede the effectiveness of the biocatalyst.

Clearly a need exists in the art for techniques that immobilize enzymes without impeding their effectiveness as biocatalysts.

### SUMMARY OF THE INVENTION

In certain embodiments, the present invention provides a biomolecular condensate comprising a plurality of cross-linked, immobilized fusion proteins. In certain embodiments, the fusion proteins have at least one first protein of interest operably linked to at least one heterologous intrinsically disordered region (IDR) sequence; said crosslinked biomolecular condensate lacking liquid-like properties. In certain embodiments, the first protein of interest is an enzyme which retains catalytic activity after crosslinking. In certain embodiments, the first protein of interest has at least one reactive surface residue. In certain embodiments, the first protein of interest is an alcohol dehydrogenase, ketoreductase, alcohol oxidase, transaminase, monooxygenase, acylase, lipase, esterase, thioesterase, caboxylic acid reductase, aldolase, amine dehydrogenase, polymerase, lactase, or amylase. In certain embodiments, the first protein of interest is a ω-transaminase.

In certain embodiments, the particle comprises a second, third or fourth protein of interest operably linked to an IDR; said second, third or fourth protein of interest being different enzymes from said first protein of interest and retaining catalytic activity; said multiple enzymes forming a coordinated biocatalytic cascade that efficiently catalyze multistep enzymatic reactions. In certain embodiments, the IDR drives phase separation. In certain embodiments, the IDR is RGG or PGL3.

In certain embodiments, the fusion proteins are cross-linked by the formation of an isopeptide bond or a disulfide bond. In certain embodiments, wherein the fusion proteins are cross-linked using glutaraldehyde, BS3, bis-maleimide, or other crosslinking reagents with reactivity towards amines or sulfhydryls. In certain embodiments, the plurality of fusion proteins present in the particle are not identical. In certain embodiments, the biomolecular condensate has a diameter of between about 0.25 μm-about 20 μm, or between about 0.25 μm-4 μm.

In certain embodiments, solid supports comprising the biomolecular condensates described herein are provided.

In certain embodiments, the present invention comprises methods for producing an immobile, biologically active biomolecular condensate particle comprising at least one IDR operably linked to a first enzyme. In certain embodiments, the method comprises a) providing a cell comprising a fusion protein having at least one protein of interest and at least one intrinsically disordered region (IDR) sequence; b) lysing said cell thereby forming a lysate; c) collecting the fusion protein from said lysate using liquid-liquid phase separation (LLPS) and/or chromatography; d) contacting the fusion protein of step c) with a crosslinking agent under conditions suitable for crosslinking to occur while maintaining catalytic activity of said enzyme such that a particle is formed containing crosslinked fusion protein; and, optionally e) isolating said plurality of crosslinked IDR-catalytically active enzyme fusion protein containing particles. In certain embodiments, the first protein of interest is an alcohol dehydrogenase, ketoreductase, alcohol oxidase, transaminase, monooxygenase, acylase, lipase, esterase, thioesterase, caboxylic acid reductase, aldolase, amine dehydrogenase, polymerase, lactase, or amylase. In certain embodiments, the first protein of interest is a ω-transaminase.

In certain embodiments, the conditions comprise modulating at least one parameter selected from cross-linking agent concentrations, cross-linking temperature, and time period for cross linking to occur. In certain embodiments, the conditions comprise 0-4° C. for 1 hour. In certain embodiments, the cross linking agent is selected from the group consisting of glutaraldehyde, BS3, bis-maleimide, or other crosslinking reagents with reactivity towards amines or sulfhydryls. In certain embodiments, the fusion proteins are cross-linked by the formation of an isopeptide bond.

In certain embodiments, the at least two fusion proteins comprise catalytically active enzymes that are not identical, and form a coordinated multi-enzyme biocatalytic cascade that efficiently catalyzes multistep enzymatic reactions. In certain embodiments, the crosslinking conditions generate a crosslinked biomolecular condensate with a diameter of between about 0.25 μm-about 20 μm, or between about 0.25 μm-4 μm. In certain embodiments, the chromatography is Ni-NTA affinity chromatography. In certain embodiments, the cells are lysed by sonication. In certain embodiments, the cross-linking reaction is quenched prior to isolating the fusion protein, In certain embodiments, the cross-linking reaction is quenched by addition of tris(hydroxymethyl)aminomethane (Tris).

In certain embodiments, the methods further comprise centrifuging the lysate prior to step d). In certain embodiments, the methods further comprise detecting the formation of the crosslinked fusion proteins. In certain embodiments, the crosslinked, enzymatically active biomolecular condensate does not exhibit liquid-like properties.

Still other aspects and advantages of these compositions and methods are readily apparent and described further in the following detailed description of the invention.

### BRIEF DESCRIPTION OF THE DRAWINGS

FIG. 1: A graphical representation showing the methods for immobilizing enzymes using cross-linked biomolecular condensates.

FIG. 2A-2B: General schematic for carrier-free enzyme immobilization techniques. (FIG. 2A) General method for preparing cross-linked enzyme aggregates (CLEAs) (adapted from Yamaguchi et al. Techniques for Preparation of Cross-Linked Enzyme Aggregates and Their Applications in Bioconversions. Catalysts 2018, 8, 174.); (FIG. 2B) Proposed method for immobilizing enzymes by engineering enzyme-RGG fusion proteins, inducing phase separation into biomolecular condensates, and cross-linking condensates.

FIG. 3A-3F: Cross-linked GFP-RGG-RGG condensates do not exhibit liquid-like properties. (FIG. 3A) Untreated and (FIG. 3D) cross-linked GFP-(RGG) 2 condensates were photobleached and the fluorescence recovery was monitored by confocal microscopy. (FIG. 3E) Cross-linked GFP-(RGG) 2 remained photobleached for the full ˜58 s. (FIG. 3B) Untreated GFP-(RGG) 2 condensates recovered some fluorescence over the course of ˜58 seconds whereas cross-linked condensates showed very little recovery. (FIG. 3C) Untreated RGG-GFP-RGG condensates placed on a glass surface have a much lower contact angle (average=˜65.4°) compared to (FIG. 3F) cross-linked condensates (average=) 131.9°. Scale bar=5 μm.

FIG. 4A-4D: Cross-linked RGG-GFP-RGG condensates can withstand a wider range of conditions. Untreated RGG-GFP-RGG condensates (FIG. 4A) will dissolve in the presence of 150 mM NaCl, 2.5 M imidazole, pH 8.0 buffer (FIG. 4B). However, cross-linked RGG-GFP-RGG condensates (FIG. 4C) resist dissolution under the same conditions (FIG. 4D). Scale bar=5 μm.

FIG. 5A-5B: SDS-PAGE and turbidity assay of cross-linked RGG-GFP-RGG samples shows evidence of multimerization. (FIG. 5A) As the RGG-GFP-RGG concentration in solution increases, the protein is more likely to phase separate, resulting in a higher occurrence of multimerization after the cross-linker is added. (FIG. 5B) Turbidity assay of cross-linked RGG-GFP-RGG samples. [RGG-GFP-RGG]=˜10 μM; 150 mM NaCl; 50 mM Borate; 20 mM Tris.

FIG. 6A-6B: Images showing that cross-linked condensates are permeable and can enrich molecules.

FIG. 7: Images showing that microparticle size can be controlled by allowing droplets to coalesce before crosslinking and/or increasing initial protein concentration.

FIG. 8A-8B: Validation of RGG-SpyCatcher003-RGG cross-linking. (FIG. 8A) When RGG-SpyCatcher003-RGG (RSCR) samples are normally run in SDS-PAGE, the protein will appear as a singular band around 56 kDa (Lanes 2 and 3). After cross-linking, the 56 kDa disappears and a large portion of the sample fails to migrate through the polyacrylamide gel (Lanes 4 and 5). (FIG. 8B) Turbidity assays performed on untreated and cross-linked RGG-SpyCatcher-RGG samples show that while untreated RSCR samples dramatically increase in turbidity at lower temperatures due to phase separation, cross-linked samples remain turbid over the tested temperature range.

FIG. 9A-9C: RSCR particles can be conjugated with SpyTagged proteins after cross-linking RGG-SpyCatcher003-RGG condensates were cross-linked using BS3 to generate solid particles (FIG. 9A). The RSCR particles were spun down, washed, and added to solutions containing GFP-SpyTag003 (FIG. 9B) or (as a control) SYNZIP2-GFP (FIG. 9C). Scale bar=5 μm.

FIG. 10A-10C: RGG-mCherry-RGG colocalizes into RGG-GFP-RGG condensates (FIG. 10A) RGG-GFP-RGG with a concentration of 10 μM and (FIG. 10B) RGG-mCherry-RGG with a concentration of 5 μM co-phase separated to form condensates containing both RGG fusion proteins (FIG. 10C). Scale bar=5 μm

FIG. 11A-11F: Phase separating proteins combined with surfactant proteins can co-localize and be crosslinked to create particles with microarchitecture. (RGG) 2 can be mixed with MBP-GFP-RGG to form core-shell condensates (FIG. 11A). When these core-shell condensates are photobleached (FIG. 11B), their fluorescence will recover (FIG. 11C) because of other molecules diffusing to the condensate surface from the environment. Core-shell (RGG)₂+MBP-GFP-RGG condensates can be cross-linked (FIG. 11D). However, these cross-linked condensates do not recover fluorescence (FIG. 11E, 11F), indicating that they have been solidified. Scale bar=5 μm

FIG. 12A-12C: Graphical representation of ωTA reactions including (FIG. 12A) kinetic resolution; (FIG. 12B) Asymmetric synthesis; and (FIG. 12C) the biocatalytic synthesis of Sitagliptin. FIGS. 12A-12B are adapted from Koszelewski, Dominik, et al. “ω-Transaminases for the synthesis of non-racemic α-chiral primary amines.” Trends in biotechnology 28.6 (2010): 324-332. FIG. 12C is adapted from Song, J. J., et al. “9.3 Industrial applications of asymmetric synthesis: asymmetric synthesis as an enabler of green chemistry.” Comprehensive Chirality (2012): 46-72.

FIG. 13: Images identifying BsADH-RGG and RGG-BsADH cross-linked enzyme condensates.

FIG. 14A-14B: Fluorescent Images showing Fluorescein-NAD⁺ partitioning into cross-linked BsADH condensates. After photobleaching, a large portion of the fluorescein-NAD does not recover, indicating that much of the molecules are bound to the enzyme.

FIG. 15: Tabular and graphical representation of cross-linked BsADH condensate catalytic activity.

FIG. 16: Tabular and graphical representation of cross-linked BsADH condensate relative activities at different temperatures.

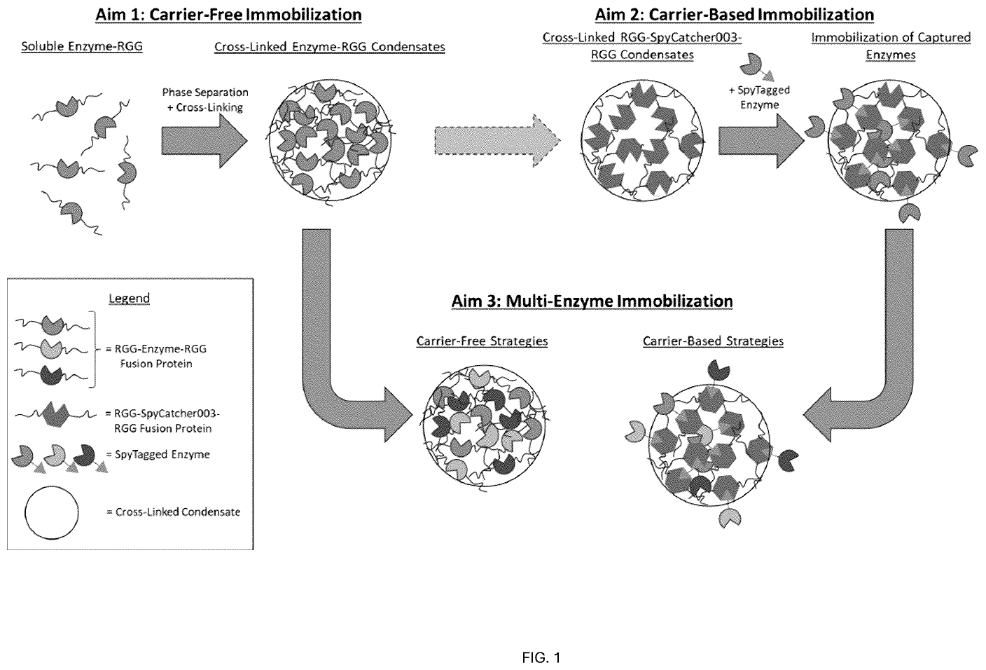

### DETAILED DESCRIPTION OF THE INVENTION

Natural biomolecular condensates are used by cells to sequester biomolecules and regulate reaction kinetics. Though biomolecular condensates are normally sensitive to environmental cues, they can be cross-linked to form the basis of novel biomaterials.

Using this as a basis, we have developed innovative methods of enzyme immobilization by crosslinking biomolecular condensates. In an exemplary embodiment, the invention disclosed herein focuses on immobilizing transaminases, which catalyze the transfer of an amino group from an amino donor substrate to an amino acceptor (FIG. 12). In certain embodiments, the amino donor is a substrate with at least one methylene group between the α-carbon and the amino group. This embodiment shows that using crosslinked biomolecular condensates to immobilize enzymes yields superior catalytically active biomaterials for biocatalytic processes.

This novel method for enzyme immobilization provides an innovative and facile method for immobilization that utilizes mild conditions and that can be easily tuned depending on the enzymes being immobilized. After inducing phase-separation and crosslinking the enzymes, or by capturing them into already crosslinked condensates, the enzymes remain catalytically active, are capable of withstanding a wider range of conditions than soluble enzymes, and can be easily separated from solution by centrifugation and/or filtration.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ˜20% or ˜10%, more preferably ˜5%, even more preferably ˜1%, and still more preferably ˜0.1% from the specified value, as such variations are appropriate to perform the disclosed methods.

### Biomolecular Condensates and Fusion Proteins

Provided herein are immobilized biomolecular condensates and fusion proteins for use in biocatalysis. In certain embodiments, the fusion proteins comprise at least one intrinsically disorder region (IDR) and at least one polypeptide of interest. In certain embodiments, the IDR and the polypeptide of interest originate from different peptides.

The term “intrinsically disordered region” or “IDR” refers to segments within proteins that lack a fixed tertiary structure and can exist as a dynamic ensemble of interconverting conformations. These IDRs are often characterized by amino acid sequences biased towards hydrophobic amino acids and charged, hydrophilic amino acids. The low amino acid complexity within these IDRs allows for proteins to engage in multivalent, transient interactions with various biomolecules such as other proteins, RNA, and lipids. For this reason, researchers have found that many proteins with IDRs play important regulatory functions in processes such as differentiation, transcription regulation, and DNA condensation.

Many IDRs are highly enriched in freely accessible post-translational modifications sites, which allows for cells to exert fine regulatory control over these proteins. Some IDRs lack stable secondary structures, which allows them to be used as reversible sensors and initiate signaling pathways due to their multivalent interactions. Because of the low energy barriers to binding and dissociation along with the opportunity to contain multiple interaction motifs, IDRs can interact with diverse array of targets. In certain embodiments, the IDR drives phase separation and facilitate the formation of droplets, which tend not to form in the absence of the IDR. Exemplary IDRs include, without limitation, RGG (derived from the N-terminal disordered domain of the C. elegans LAF-1 protein), PGL-3, FUS, and the IDRs identified at original.disprot.org/view_protein.php, which is incorporated herein by reference.

Additional IDRs can be identified using known protein interaction databases or prediction algorithms (such as DISOPRED3, ANCHOR, ANCHOR2, IUPred2, alpha-MoRFpred, MoRFpred, fMoRFpred, and MoRFCHiBi), each of which is incorporated by reference herein.

The term “IDR-containing proteins” or “IDPs” refer to proteins that contain at least one IDR. The protein-protein interactions of IDPs cause the formation of biomolecular condensates.

The term “biomolecular condensate” refers to two- and three-dimensional compartments in eukaryotic cells that concentrate specific collections of molecules without an encapsulating lipid-based membrane. Additionally, in the right conditions a biomolecular condensate can form outside of cells. Condensate formation has emerged as a fundamental mechanism for the organization of biomolecules within the nucleus and cytosol and at membranes. Many condensates behave as dynamic liquids and appear to form through liquid-liquid phase separation (LLPS) driven by weak, multivalent interactions between macromolecules. There are numerous manifestations of this multivalency, including sticky ultra-weak interactions between intrinsically disordered proteins (IDPs) and arrays of modular protein domains. Specific interactions, such as interactions between modular binding domains and nucleic acid base pairing, weaker interactions between intrinsically disorder regions, and nonspecific interactions, such as electrostatic interactions and hydrophobic interactions, influence condensate formation and composition.

Representative and recognized biomolecular condensates include PML nuclear bodies, P-bodies, stress granules, the nucleolus, and two-dimensional membrane localized LAT and nephrin clusters. Individual condensates can contain hundreds of distinct molecular components. In certain embodiments, the biomolecular condensate can comprise multiple copies of a single polypeptide or fusion peptide.

This invention relates to covalently crosslinking biomolecular condensates that contain enzymes to produce immobilized enzyme particles. By modifying the cross-linking conditions, the size of the biomolecular condensate particles can be controlled. In certain embodiments, the conditions comprise modulating at least one of cross-linking agent concentrations, cross-linking temperature, and time. In certain embodiments, the conditions comprise 0° C. for 1 hour. In certain embodiments, the particle diameter is larger than 0.25 μm. In certain embodiments, the particle diameter is smaller than 4 μm. In certain embodiments, the particle diameter is between 0.25 μm and 4 μm. In certain embodiments, the particle diameter is smaller than 20 μm. In certain embodiments, the particle diameter is between 0.25 μm and 20 μm. In certain embodiments, the particle diameter is between 0.5 μm and 3.4 μm. In certain embodiments, the particle diameter is between 0.55 μm and 3.38 μm. In certain embodiments, the particle diameter is at least about 0.25 μm, about 0.30 μm, about 0.35 μm, about 0.40 μm, about 0.45 μm, about 0.50 μm, about 0.55 μm, about 0.60 μm, about 0.65 μm, about 0.70 μm, about 0.75 μm, about 0.80 μm, about 0.85 μm, about 0.90 μm, about 0.95 μm, about 1.00 μm, about 1.05 μm, about 1.10 μm, about 1.15 μm, about 1.20 μm, about 1.25 μm, about 1.30 μm, about 1.35 μm, about 1.40 μm, about 1.45 μm, about 1.50 μm, about 1.55 μm, about 1.60 μm, about 1.65 μm, about 1.70 μm, about 1.75 μm, about 1.80 μm, about 1.85 μm, about 1.90 μm, about 1.95 μm, about 2.00 μm, about 2.05 μm, about 2.10 μm, about 2.15 μm, about 2.20 μm, about 2.25 μm, about 2.30 μm, about 2.35 μm, about 2.40 μm, about 2.45 μm, about 2.50 μm, about 2.55 μm, about 2.60 μm, about 2.65 μm, about 2.70 μm, about 2.75 μm, about 2.80 μm, about 2.85 μm, about 2.90 μm, about 2.95 μm, about 3.00 μm, about 3.05 μm, about 3.10 μm, about 3.15 μm, about 3.20 μm, about 3.25 μm, about 3.30 μm, about 3.35 μm, about 3.40 μm, about 3.45 μm, about 3.50 μm, about 3.55 μm, about 3.60 μm, about 3.65 μm, about 3.70 μm, about 3.75 μm, about 3.80 μm, about 3.85 μm, about 3.90 μm, about 3.95 μm, about 4.00 μm, about 4.05 μm, about 4.10 μm, about 4.15 μm, about 4.20 μm, about 4.25 μm, about 4.30 μm, about 4.35 μm, about 4.40 μm, about 4.45 μm, about 4.50 μm, about 4.55 μm, about 4.60 μm, about 4.65 μm, about 4.70 μm, about 4.75 μm, about 4.80 μm, about 4.85 μm, about 4.90 μm, about 4.95 μm, about 5.00 μm, about 5.05 μm, about 5.10 μm, about 5.15 μm, about 5.20 μm, about 5.25 μm, about 5.30 μm, about 5.35 μm, about 5.30 μm, about 5.35 μm, about 5.40 μm, about 5.45 μm, about 5.50 μm, about 5.55 μm, about 5.60 μm, about 5.65 μm, about 5.70 μm, about 5.75 μm, about 5.80 μm, about 5.85 μm, about 5.90 μm, about 5.95 μm, about 6.00 μm, about 6.05 μm, about 6.10 μm, about 6.15 μm, about 6.20 μm, about 6.25 μm, about 6.30 μm, about 6.35 μm, about 6.40 μm, about 6.45 μm, about 6.50 μm, about 6.55 μm, about 6.60 μm, about 6.65 μm, about 6.70 μm, about 6.75 μm, about 6.80 μm, about 6.85 μm, about 6.90 μm, about 6.95 μm, about 7.00 μm, about 7.05 μm, about 7.10 μm, about 7.15 μm, about 7.20 μm, about 7.25 μm, about 7.30 μm, about 7.35 μm, about 7.40 μm, about 7.45 μm, about 7.50 μm, about 7.55 μm, about 7.60 μm, about 7.65 μm, about 7.70 μm, about 7.75 μm, about 7.80 μm, about 7.85 μm, about 7.90 μm, about 7.95 μm, about 8.00 μm, about 8.05 μm, about 8.10 μm, about 8.15 μm, about 8.20 μm, about 8.25 μm, about 8.30 μm, about 8.35 μm, about 8.40 μm, about 8.45 μm, about 8.50 μm, about 8.55 μm, about 8.60 μm, about 8.65 μm, about 8.70 μm, about 8.75 μm, about 8.80 μm, about 8.85 μm, about 8.90 μm, about 8.95 μm, about 9.00 μm, about 9.05 μm, about 8.10 μm, about 9.15 μm, about 9.20 μm, about 9.25 μm, about 9.30 μm, about 9.35 μm, about 9.40 μm, about 9.45 μm, about 9.50 μm, about 9.55 μm, about 9.60 μm, about 9.65 μm, about 9.60 μm, about 9.65 μm, about 9.70 μm, about 9.75 μm, about 9.80 μm, about 9.85 μm, about 9.90 μm, about 9.95 μm, about 10.00 μm, about 10.05 μm, about 10.10 μm, about 10.15 μm, about 10.20 μm, about 10.25 μm, about 10.30 μm, about 10.35 μm, about 10.40 μm, about 10.45 μm, about 10.50 μm, about 10.55 μm, about 10.60 μm, about 10.65 μm, about 10.70 μm, about 10.75 μm, about 10.80 μm, about 10.85 μm, about 10.90 μm, about 10.95 μm, about 11.00 μm, about 11.05 μm, about 11.10 μm, about 11.15 μm, about 11.20 μm, about 11.25 μm, about 11.30 μm, about 11.35 μm, about 11.40 μm, about 11.45 μm, about 11.50 μm, about 11.55 μm, about 11.60 μm, about 11.65 μm, about 11.70 μm, about 11.75 μm, about 11.80 μm, about 11.85 μm, about 11.90 μm, about 11.95 μm, about 12.00 μm, about 12.05 μm, about 12.10 μm, about 12.15 μm, about 12.20 μm, about 12.25 μm, about 12.30 μm, about 12.35 μm, about 12.40 μm, about 12.45 μm, about 12.50 μm, about 12.55 μm, about 12.60 μm, about 12.65 μm, about 12.70 μm, about 12.75 μm, about 12.80 μm, about 12.85 μm, about 12.90 μm, about 12.95 μm, about 13.00 μm, about 13.05 μm, about 13.10 μm, about 13.15 μm, about 13.20 μm, about 13.25 μm, about 13.30 μm, about 13.35 μm, about 13.40 μm, about 13.45 μm, about 13.50 μm, about 13.55 μm, about 13.60 μm, about 13.65 μm, about 13.70 μm, about 13.75 μm, about 13.80 μm, about 13.85 μm, about 13.90 μm, about 13.95 μm, about 14.00 μm, about 14.05 μm, about 14.10 μm, about 14.15 μm, about 14.20 μm, about 14.25 μm, about 14.30 μm, about 14.35 μm, about 14.40 μm, about 14.45 μm, about 14.50 μm, about 14.55 μm, about 14.60 μm, about 14.65 μm, about 14.70 μm, about 14.75 μm, about 14.80 μm, about 14.85 μm, about 14.90 μm, about 14.95 μm, about 15.00 μm, about 15.05 μm, about 15.10 μm, about 15.15 μm, about 15.20 μm, about 15.25 μm, about 15.30 μm, about 15.35 μm, about 15.30 μm, about 15.35 μm, about 15.40 μm, about 15.45 μm, about 15.50 μm, about 15.55 μm, about 15.60 μm, about 15.65 μm, about 15.70 μm, about 15.75 μm, about 15.80 μm, about 15.85 μm, about 15.90 μm, about 15.95 μm, about 16.00 μm, about 16.05 μm, about 16.10 μm, about 16.15 μm, about 16.20 μm, about 16.25 μm, about 16.30 μm, about 16.35 μm, about 16.40 μm, about 16.45 μm, about 16.50 μm, about 16.55 μm, about 16.60 μm, about 16.65 μm, about 16.70 μm, about 16.75 μm, about 16.80 μm, about 16.85 μm, about 16.90 μm, about 16.95 μm, about 17.00 μm, about 17.05 μm, about 17.10 μm, about 17.15 μm, about 17.20 μm, about 17.25 μm, about 17.30 μm, about 17.35 μm, about 17.40 μm, about 17.45 μm, about 17.50 μm, about 17.55 μm, about 17.60 μm, about 17.65 μm, about 17.70 μm, about 17.75 μm, about 17.80 μm, about 17.85 μm, about 17.90 μm, about 17.95 μm, about 18.00 μm, about 18.05 μm, about 18.10 μm, about 18.15 μm, about 18.20 μm, about 18.25 μm, about 18.30 μm, about 18.35 μm, about 18.40 μm, about 18.45 μm, about 18.50 μm, about 18.55 μm, about 18.60 μm, about 18.65 μm, about 18.70 μm, about 18.75 μm, about 18.80 μm, about 18.85 μm, about 18.90 μm, about 18.95 μm, about 19.00 μm, about 19.05 μm, about 19.10 μm, about 19.15 μm, about 19.20 μm, about 19.25 μm, about 19.30 μm, about 19.35 μm, about 19.40 μm, about 19.45 μm, about 19.50 μm, about 19.55 μm, about 19.60 μm, about 19.65 μm, about 19.60 μm, about 19.65 μm, about 19.70 μm, about 19.75 μm, about 19.80 μm, about 19.85 μm, about 19.90 μm, about 19.95 μm, about 20.00 μm.

The term “cross-link” or “crosslink” or “Cross link” refers to a bond, or a short sequence of bonds, that links one polymer chain to another. In certain embodiments, the bond is a covalent bond.

In certain embodiments, biomolecular condensates comprising the cross-linked fusion proteins, described herein, do not exhibit liquid-like properties. The term “liquid-like properties” refers to the ability of biomolecular condensates—prior to their crosslinking—to coalesce, wet, flow, or dissolve/condense similar to a viscous liquid. In contrast, in certain embodiments, after crosslinking, the biomolecular condensates do not exhibit such liquid-like properties and instead exhibit solid-like properties.

As used herein, unless otherwise specified, the terms “peptide” and “polypeptide” refer to polymer compounds of two or more amino acids joined through the main chain by peptide amide bonds (—C(O)NH—). The term “peptide” typically refers to short amino acid polymers (e.g., chains having fewer than 30 amino acids), whereas the term “polypeptide” typically refers to longer amino acid polymers (e.g., chains having more than 30 amino acids).

As used herein, the term “polypeptide of interest” refers to a polypeptide to be produced and is intended to encompass any protein or peptide encoded by the genome of a host cell or expressed in the host cell, covering polypeptides unknown for activity thereof as well as proteins and/or peptides having desired activities in vivo (e.g., activities of preventing, alleviating, and/or treating specific diseases, or of substituting for biologically necessary substances). The polypeptide of interest may be at least one selected from the group consisting of polypeptides located in the cytoplasm, polypeptides located in the cell membrane, and extracellularly excreted polypeptides. For example, the polypeptide of interest may be at least one selected from the group consisting of enzymes, hormones, growth factors, receptors, transport polypeptides, immune polypeptides (generic name for polypeptides produced in immune cells), signaling polypeptides, and biologically structural polypeptides. In certain embodiments, the protein of interest is an enzyme. In certain embodiments, the protein of interest is a transaminase. In certain embodiments, the transaminase is a ω-TA. In certain embodiments, the protein of interest comprises at least one reactive surface residue. In certain embodiments, the protein of interest is a fragment of the naturally occurring protein.

In an embodiment, exemplary polypeptides of interest include, without limitation: enzymes including hydrolases (e.g., proteinases, phosphatases, etc.), oxidoreductases, transferases (e.g. kinases and transaminases, which transfer amino and phosphoryl groups, respectively), lyases, ligases, and isomerases; and hormones, growth factors, receptors, transport polypeptides, immune polypeptides, cytokines (e.g., tumor necrosis factors and TNF-related apoptosis-inducing ligand (TRAIL)), growth factors (e.g., colony stimulating factor, CSF), various signaling polypeptides, and other biologically structural polypeptides.

In certain embodiments, the protein of interest is an enzyme. In certain embodiments, the enzyme is an alcohol dehydrogenase, ketoreductase, alcohol oxidase, transaminase, monooxygenase, acylase, lipase, esterase, thioesterase, caboxylic acid reductase, aldolase, amine dehydrogenase, polymerase, lactase, or amylase. Additional proteins of interest include the biocatalysts identified in Bell, E. L., Finnigan, W., France, S. P. et al. Biocatalysis. Nat Rev Methods Primers 1, 46 (2021). doi.org/10.1038/s43586-021-00044-z which is incorporated by reference herein.

As used herein, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein). A peptide or polypeptide that is “not a fragment of a preexisting protein” is an amino acid chain that is not a subsequence of a protein (e.g., natural or synthetic) that was in physical existence prior to design and/or synthesis of the peptide or polypeptide.

As used herein, the terms “fusion,” “fusion polypeptide,” and “fusion protein” refer to a chimeric protein containing a first protein or polypeptide of interest joined to a second different peptide, polypeptide, or protein. In certain embodiments, the fusion protein contains a linker sequence between the first and second peptides.

In the present context, the term “linker” refers to a connection between two protein coding sequences or their protein products. Linkers comprise a stretch of contiguous nucleic acids or amino acids, which holds at least one cleavage site that enables separation of the genes or their products through cleavage of the linker.

As used herein, the terms “conjugated” and “conjugation” refer to the covalent attachment of two molecular entities (e.g., post-synthesis and/or during synthetic production). The attachment of a peptide or small molecule tag to a protein or small molecule, chemically (e.g., “chemically” conjugated) or enzymatically, is an example of conjugation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid residues may be divided into classes based on common side chain properties, for example: polar positive (or basic) (e.g., histidine (H), lysine (K), and arginine (R)); polar negative (or acidic) (e.g., aspartic acid (D), glutamic acid (E)); polar neutral (e.g., serine(S), threonine (T), asparagine (N), glutamine (Q)); non-polar aliphatic (e.g., alanine (A), valine (V), leucine (L), isoleucine (I), methionine (M)); non-polar aromatic (e.g., phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

“Conservative amino acid substitution” refers to the interchange of a residue having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Nonpolar Amino Acid” refers to an amino acid having a side chain that is uncharged at physiological pH, that is not polar and that is generally repelled by aqueous solution. Examples of genetically encoded hydrophobic amino acids include Ala, Ile, Leu, Met, Trp, Tyr, and Val. Examples of non-genetically encoded nonpolar amino acids include t-BuA, Cha, and Nle.

“Aromatic Amino Acid” refers to a nonpolar amino acid having a side chain containing at least one ring having a conjugated pi-electron system (aromatic group). The aromatic group may be further substituted with substituent groups such as alkyl, alkenyl, alkynyl, hydroxyl, sulfonyl, nitro, and amino groups, as well as others. Examples of genetically encoded aromatic amino acids include phenylalanine, tyrosine, and tryptophan. Commonly encountered non-genetically encoded aromatic amino acids include phenylglycine, 2-naphthylalanine, β-2-thienylalanine, 3-benzothiazol-2-yl-alanine, 1,2,3,4-tetrahydroisoquinoline-3-carboxylic acid, 4-chlorophenylalanine, 2-fluorophenylalanine, 3-fluorophenylalanine, and 4-fluorophenylalanine.

“Aliphatic Amino Acid” refers to a nonpolar amino acid having a saturated or unsaturated straight chain, branched or cyclic hydrocarbon side chain. Examples of genetically encoded aliphatic amino acids include Ala, Leu, Val, and Ile. Examples of non-encoded aliphatic amino acids include Nle.

“Polar Amino Acid” refers to a hydrophilic amino acid having a side chain that is charged or uncharged at physiological pH and that has a bond in which the pair of electrons shared in common by two atoms is held more closely by one of the atoms. Polar amino acids are generally hydrophilic, meaning that they have an amino acid having a side chain that is attracted by aqueous solution. Examples of genetically encoded polar amino acids include asparagine, cysteine, glutamine, lysine, and serine. Examples of non-genetically encoded polar amino acids include citrulline, homocysteine, N-acetyl lysine, and methionine sulfoxide.

“Acidic Amino Acid” refers to a hydrophilic amino acid having a side chain pKₐ value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pKₐ value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include ornithine, 2,3-diaminopropionic acid, 2,4-diaminobutyric acid, and homoarginine.

“Ionizable Amino Acid” refers to an amino acid that can be charged at a physiological pH. Such ionizable amino acids include acidic and basic amino acids, for example, D-aspartic acid, D-glutamic acid, D-histidine, D-arginine, D-lysine, D-hydroxylysine, D-ornithine, L-aspartic acid, L-glutamic acid, L-histidine, L-arginine, L-lysine, L-hydroxylysine, or L-ornithine.

As will be appreciated by those having skill in the art, the above classifications are not absolute. Several amino acids exhibit more than one characteristic property, and can therefore be included in more than one category. For example, tyrosine has both a nonpolar aromatic ring and a polar hydroxyl group. Thus, tyrosine has several characteristics that could be described as nonpolar, aromatic and polar. However, the nonpolar ring is dominant and so tyrosine is generally considered to be nonpolar. Similarly, in addition to being able to form disulfide linkages, cysteine also has nonpolar character. Thus, while not strictly classified as a hydrophobic or nonpolar amino acid, in many instances cysteine can be used to confer hydrophobicity or nonpolarity to a peptide.

In some embodiments, polar amino acids contemplated by the present invention include, for example, arginine, asparagine, aspartic acid, cysteine, glutamic acid, glutamine, histidine, homocysteine, lysine, hydroxylysine, ornithine, serine, threonine, and structurally related amino acids. In one embodiment the polar amino is an ionizable amino acid such as arginine, aspartic acid, glutamic acid, histidine, hydroxylysine, lysine, or ornithine.

Examples of polar or nonpolar amino acid residues that can be utilized include, for example, alanine, valine, leucine, methionine, isoleucine, phenylalanine, tryptophan, tyrosine, and the like.

The term “nucleic acid” refers to a single or double-stranded polymer of deoxyribonucleotide or ribonucleotide bases read from the 5′ to the 3′ end.

### Vectors and Host Cells

Also provided herein are vectors encoding the fusion proteins described herein and host cells comprising the fusion proteins and/or biomolecular condensates described herein.

The term “vector” relates to a single or double stranded linear or circular nucleic acid molecule that can be infected, transfected or transformed into cells and replicate independently or within the host cell genome. A linear or circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

The introduced nucleic acid may or may not be integrated (covalently linked) into nucleic acid of the recipient cell or organism. In plant cells, for example, the introduced nucleic acid may be maintained as an episomal element or independent replicon, such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “encoding” refers to a polynucleotide sequence encoding one or more amino acids. The term does not require a start or stop codon. An amino acid sequence can be encoded in any one of six different reading frames provided by a double-stranded polynucleotide sequence. In some variations, encoding sequences further include a start and/or a stop codon.

Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include polyethylene glycol mediated protoplast transformation, lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified and that retains the modification, such as a daughter cell. Thus, for example, recombinant cells express genes that are not found within the native (nonrecombinant) form of the cell or express native genes that are otherwise abnormally expressed, under-expressed or not expressed at all.

### Solid Supports

Also provided herein are solid supports comprising the fusion proteins or biomolecular condensates described herein.

The term “solid support” or “solid matrix” as used herein refers to any format, such as beads, microparticles, a microarray, the surface of a microtitration well or a test tube, a dipstick or a filter. The material of the matrix may be polypeptides, or may include other materials, e.g. polystyrene, cellulose, latex, nitrocellulose, nylon, polyacrylamide, dextran or agarose.

### Methods of Manufacture

Also provided herein are methods of producing the biomolecular condensates described herein. In certain embodiments, the methods comprise a) providing a cell comprising at least one of the fusion proteins described herein; b) lysing the cell, thereby forming a lysate; c) collecting the fusion protein from said lysate using liquid-liquid phase separation (LLPS) and/or chromatography; and d) contacting the fusion protein of step c) with a cross-linking agent under conditions suitable for cross linking to occur while maintaining catalytic activity of said enzyme such that a fusion protein containing cross linked particle is formed. In certain embodiments, the methods further comprise isolating said cross linked IDR-catalytically active enzyme fusion protein containing particle. In certain embodiments, the biomolecular condensate produced by this method is solid-like and biologically active. In certain embodiments, the cell is provided by transforming or transfecting the cell with a vector that encodes the biomolecular condensate. In certain embodiments, the methods further comprise quenching the cross-linking reaction prior to isolating the fusion protein. In certain embodiments, the reaction is quenched by adding tris(hydroxymethyl)aminomethane (Tris).

The term “lysis” or “lysed” refers to the breakdown of a cell caused by damage to its cell wall and/or plasma membrane. Lysis can be caused by chemical, physical, or viral means. The choice of cell lysis method generally depends on the type of cells, volume, and sensitivity of the proteins being extracted. The skilled artisan is capable of determining the preferred method for lysing the cells based on these properties. In certain embodiments, multiple methods of lysing the cell, including, for example, both physical and chemical means, can be used together to ensure full lysis of the cell. The product of a lysed cell is known as the lysate.

In physical disruption methods, the cell wall and/or membrane is physically broken down by shear or external forces to release the cellular components. Several methods are commonly used to physically lyse cells to extract proteins, including without limitation, mechanical disruption, liquid homogenization, sonication, freeze/thaw cycles, and manual grinding. In certain embodiments, the cells are lysed using sonication.

Cells can also be lysed using chemical means. Detergent or solution-based lysis is generally milder and easier than physical disruption. These solutions lyse the cells by breaking the lipid barrier by solubilizing proteins and disruption of lipid-lipid, protein-protein, and protein-lipid interactions. Numerous chemicals that are capable of lysing cells are known to the skilled artisan. By analyzing the properties of the protein to be extracted, the skilled artisan is capable of determining the preferred chemicals for lysing the cells.

The term “liquid-liquid phase separation” or “LLPS” refers to a thermodynamic phenomenon where a heterogeneous mixture of poorly miscible molecules demixes into separate phases. Each of these phases will have different concentrations of different components. Due to the formation of the biomolecular condensates described herein, the fusion proteins can be separated from the rest of the cell using LLPS. In certain embodiments, LLPS can be performed by waiting for the cellular components to separate over time. Additionally, other methods such as affinity chromatography can be used to further purify the proteins. In certain embodiments, Ni-NTA affinity chromatography is used to purify the proteins. In certain embodiments, non-chromatographic methods utilizing phase separation can be used to purify the proteins. In certain embodiments, a chromatography-free transition temperature cycling protocol can be used to purify IDPs from cell lysates. If the lysate contains IDPs, phase separation can be induced to have the IDPs form biomolecular condensates by heating or cooling the lysate depending on the IDP. The biomolecular condensates are dense enough to be able to be centrifuged out of solution and form a pellet. The supernatant can be decanted and the pellet can be resuspended in buffer conditions that are unfavorable for phase separation. Subsequently, the now soluble IDP can be separated from the insoluble cell components by another round of centrifugation, allowing for a chromatography-free method of purification. Exemplary protocols can be found in Meyer and Chilkoti (2002) and Li et al. (2024) each of which is incorporated herein by reference.

The term “cross-linking agent” or “crosslinking agent” refers to a chemical compound that can connect polymer chains to each other to form a network structure through covalent chemical bonds. In general, any suitable cross-linking agent may be incorporated into the composition. In certain embodiments, the cross-linking agent is selected from a chemical cross-linking agent; such as BS3 (bissulfosuccinimidyl suberate) and related crosslinking agents (e.g. BS(PEG)5), glutaraldehyde, crosslinkers with poly(ethylene glycol) spacers, 1-ethyl-3-(3-dimethylaminopropyl), bis-maleimide crosslinkers such as BM(PEG)2 (1,8-bismaleimido-diethyleneglycol); or enzymatic cross-linking agents, such as microbial transglutaminase (MTG). Other crosslinking agents can be identified using a crosslinking selection tool, such as the tool found at https://www.thermofisher.com/us/en/home/life-science/protein-biology/protein-labeling-crosslinking/protein-crosslinking/crosslinker-selection-tool.htm, which is incorporated by reference herein. In a preferred embodiment, the crosslinking selection tool is used to find suitable crosslinkers that link amine to amine, amin-sulfhydryl, amine-carboxyl, or sulfhydryl-sulfhydryl.

The cross-linking agent, if used, may be present in an amount, e.g., up to 20 weight %, from 0.01 weight % to 20 weight %, from 0.1 weight % to 5 weight %. The term “weight %” when used in connection with cross-linking agents, refers to the (total weight of cross-linking agents)/(total weight of all components in solution)*100).

In certain embodiments, the SpyCatcher/SpyTag system is used attach polypeptides (such as enzymes) to the crosslinked condensates. In certain embodiments, the SpyCatcher/SpyTag system is selected from the SpyCatcher001/SpyTag001, SpyCatcher002/SpyTag002, or SpyCatcher003/SpyTag003. SpyCatcher system is a technology for irreversible conjugation of recombinant proteins. The peptide SpyTag (13 amino acids) spontaneously reacts with the protein SpyCatcher (12.3 kDa) to form an intermolecular isopeptide bond between the pair. Using the Tag/Catcher pair, bioconjugation can be achieved between two recombinant proteins that would otherwise be restrictive or impossible with traditional direct genetic fusion between the two proteins.

The first SpyTag system (SpyTag001/SpyTgb001) was developed by splitting and engineering the CnaB2 domain of the FbaB adhesin protein from Streptococcus pyogenes.¹⁰⁷ SpyTagged proteins react with SpyCatcher in high specificity with a second-order rate constant of 1.4×10³ M⁻¹ s⁻¹.¹⁰⁷ The primary outcome of this experiment is development of a novel protein-based carrier material for enzyme immobilization.

The “SpyTag002/SpyCatcher002” refers to the second generation SpyTag/SpyCatcher system. This system was created through phage display that enables the peptide-protein pair to react up to 12 times faster than the original pair, at a rate constant of 2.0±0.2×10⁴ M⁻¹ s⁻¹. The second generation SpyCatcher002 also has abolished self-reactivity that is present with SpyCatcher.

The “SpyTag003/SpyCatcher003” refers to the third generation of the SpyTag/SpyCatcher system. This reacts up to 400-fold faster than the original pair with a rate constant of 5.5±0.6×10⁵ M⁻¹ s⁻¹. This version is back reactive with the two previous generations of SpyTag/SpyCatcher reagents.

Table 1 provides a list of exemplary SpyCatcher reagents and Supporting Reagents that may be used with this system.

TABLE 1A
SpyCatcher Reagents

| | |
|---|---|
| Product | Description |

Monovalent Format

| | |
|---|---|
| SpyCatcher2 | SpyCatcher2 protein |
| SpyCatcher2-CYS | SpyCatcher2 with an |
| | engineered cysteine residue; use for |
| | site-specific chemical conjugation |
| | to a label of choice |
| SpyCatcher2-Biotin | SpyCatcher2 with an |
| | engineered cysteine |
| | residue conjugated to Biotin |
| SpyCatcher3 | SpyCatcher3 protein |
| SpyCatcher3-CYS | SpyCatcher3 with an |
| | engineered cysteine residue; use for |
| | site-specific conjugation to a label |
| | of choice |

Bivalent Format

| | |
|---|---|
| BiSpyCatcher2 | BiSpyCatcher2 protein |
| BiSpyCatcher2: Biotin | BiSpyCatcher2 with three |
| | engineered cysteine residues |
| | conjugated to biotin |
| BiSpyCatcher2: HRP | BiSpyCatcher2 with three |
| | engineered cysteine residues |
| | conjugated to HRP |
| BiSpyCatcher2-CYS | BiSpyCatcher2 with one |
| | engineered cysteine residue; use for |
| | site-specific conjugation to a label |
| | of choice |
| BiSpyCatcher2-CYS3 | BiSpyCatcher2 with three |
| | engineered cysteine residues; use |
| | for site-specific conjugation to a |
| | label of choice |

Ig-like Format

| | |
|---|---|
| hIgG1-FcSpyCatcher3 | SpyCatcher3 fused to the |
| | hinge region, CH2, and CH3 of |
| | human IgG1 |
| hIgG1-FcSpyCatcher3-Biotin | SpyCatcher3 fused to the |
| | hinge region, CH2, and CH3 of |
| | human IgG1 conjugated to Biotin |
| hIgG2-FcSpyCatcher3 | SpyCatcher3 fused to the |
| | hinge region, CH2, and CH3 of |
| | human IgG2 |
| hIgG3-FcSpyCatcher3 | SpyCatcher3 fused to the |
| | hinge region, CH2, and CH3 of |
| | human IgG3 |
| hIgG4-FcSpyCatcher3 | SpyCatcher3 fused to the |
| | hinge region, CH2, and CH3 of |
| | human IgG4 |
| hIgG4-Pro- | SpyCatcher3 fused to the |
| FcSpyCatcher3 | hinge region, CH2, and CH3 of |
| | human IgG4-Pro (S228P) |
| hIgA-FcSpyCatcher3 | SpyCatcher3 fused to the |
| | hinge region, CH2, and CH3 of |
| | human IgA |
| mIgG2a-FcSpyCatcher3 | SpyCatcher3 fused to the |
| | hinge region, CH2, and CH3 of |
| | mouse IgG2a |
| rbIgG-FcSpyCatcher3 | SpyCatcher3 fused to the |
| | hinge region, CH2, and CH3 of |
| | rabbit IgG |
| Human IgG1-N297Q- | SpyCatcher3 fused to the |
| Fc SpyCatcher3 | hinge region, CH2, and CH3 of |
| | human IgG1 with N297Q mutation |
| | in Fc region |
| Human IgG1-LALA- | SpyCatcher3 fused to the |
| Fc SpyCatcher3 | hinge region, CH2, and CH3 of |
| | human IgG1 with L234A, L235A |
| | mutation in Fc region |

TABLE 1B
Supporting Reagents

| Description | Clone |
|---|---|
| HuCAL Fab-F-Spy2-H Negative Control Antibody¹ | AbD18705ad |
| Anti-SpyCatcher Antibody | AbD41909kg |
| Anti-SpyTag Antibody | AbD51767kg |
| Anti-SpyTag HRP Antibody | AbD51767kg |
| SpyTag3 Peptide | — |

A description of the materials and methods used to generate the data set forth in the Figures is provided to facilitate the practice of the present invention.

### Materials and Methods

### Validation of Enzymatic Activity Preservation in Cross-Linked Enzyme-RGG Condensates

The examples below focus on the immobilization of transaminases (TAs), which facilitate the transfer of an amino group from an amino donor to an amino acceptor for chiral amino acid or amine synthesis.⁹⁸ TAs have gained prominence, especially in the pharmaceutical industry, for their high reaction yields, their stereoselectivity, and their simplified method for reductive amination compared to conventional chemical routes.⁹⁸⁻¹⁰⁰ For example, an engineered TA has been implemented in the synthesis of the anti-diabetic drug sitagliptin, leading to increases in both overall yield and productivity while reducing the total waste produced.⁹⁹ ω-TAs from Pseudomonas fluorescens (PfωTA) and/or Chromobacterium violaceum (CvωTA) were used herein. These TAs can be analyzed by previously published UV-absorbance-based, colorimetric, or fluorogenic assays, which all utilize commercially available substrates allowing for facile measurement of activity by a microplate reader.^{51,101,102} TAs from other species such as Aspergillus fumigatus can be commercially purchased and used as a reference.

TA gene fragments are purchased from a commercial source and cloned into modified pET-29a(+) plasmids containing an RGG domain to create TA-RGG fusion proteins. In certain embodiments, a charge-segregated variant of RGG may be used (see Schuster et al. (2020). The TA-RGG fusion proteins are overexpressed in BL21(DE3) E. coli cells. These cells are lysed by sonication, and the clarified protein supernatant is purified by Ni-NTA affinity chromatography. The protein fractions will be dialyzed into 100 mM HEPES buffer, pH 8.0.

To cross-link our TA-RGG condensates, glutaraldehyde is used instead of BS3. Glutaraldehyde is widely used in industrial settings as a cross-linker for enzyme immobilization due to its availability, low price, solubility in water, and stability. Like BS3, glutaraldehyde forms covalent bonds with primary amines. Phase separation of TA-RGG fusion proteins is induced by incubating samples on ice for 1 hour. A range of cross-linking parameters such as glutaraldehyde concentrations, cross-linking temperatures, and time will be tested to find the optimal conditions. At the conclusion of the cross-linking reaction, the glutaraldehyde is quenched by addition of 1 M tris(hydroxymethyl)aminomethane (Tris), and the condensates are spun down. The supernatant from this centrifugation step is saved for immobilization yield calculations.

The cross-linked condensates are washed with 25 mM HEPES, pH 8.0 buffer three times before being resuspended in 25 mM HEPES, 1 mM pyridoxal 5′-phosphate monohydrate (PLP), pH 8.0 buffer. Cross-linking is validated by SDS-PAGE and further analyzed by mass spectrometry to elucidate cross-linking locations. Protein concentrations before and after enzyme immobilization are measured using the bicinchoninic acid (BCA) assay. Activity assays are performed on soluble TA, soluble TA-RGG, cross-linked TA-RGG condensates, and a no-enzyme negative control. TAs are mixed with a solution containing 1.2 mM benzaldehyde, 500 mM L-alanine, 0.1 mM PLP, 10% DMSO in 100 mM, pH 8.5 bicarbonate buffer, and TA activity are determined by measuring the decrease in absorbance at 245 nm using a microplate reader. HPLC can also be used to analyze product formation at the conclusion of the reaction if necessary. Once optimal immobilization conditions are determined, activity assays using varying amounts of substrate can be performed on these TA constructs to determine Michaelis-Menten parameters Kₘ and Vₘₐₓ.

### Immobilization Yield

The immobilization yield will be calculated by the following equation:

$$Immobilization yeild ( \% ) = ( Amount of enzyme immobilized / Total amount of enzyme ) * 100 \% = ( ( Total amount of enzyme - Remaining amount of enzyme in supernatant ) / Total amount of enzyme ) * 100 \% \qquad ( 1 )$$

This value can compare different immobilization conditions and optimize for maximum yield. Bicinchoninic acid (BCA) assays and transaminase (TA) activity assays are performed with at least three technical replicates, and each experiment is conducted independently at least three times. For these assays, only standard curves with R²≥0.95 and experimental values in the linear range of the assay are accepted. The activity of cross-linked samples is compared to untreated samples and no enzyme negative controls using multiple T-tests with Bonferroni correction to determine statistical significance.

The Michaelis-Menten equation is:

$${v}_{0} = {V}_{max} [ S ] \qquad ( 2 )$$

$${K}_{m} + [ S ]$$

where v₀ is the initial product formation rate at a given substrate concentration,
   Vₘₐₓ is the maximum product formation rate,
   [S] is the substrate concentration, and
   Kₘ is the Michaelis constant, which represents the substrate concentration at which the reaction rate is at half-maximum.

Michaelis-Menten parameters Kₘ and Vₘₐₓ is calculated by curve fitting on a plot of [S] vs. v₀ in Python.

### Analysis of Cross-Linked RGG Condensates as a Carrier Material

The Examples below focus on testing different cross-linkers and optimizing cross-linking conditions to maximize capture efficiency in RGG-SpyCatcher-RGG (RSCR) particles. We test a number of commercially-available cross-linkers such as the widely used glutaraldehyde and longer length, primary amine-reactive cross-linkers like BS3, poly(ethylene glycol) diglycidyl ether, BS(PEG)5, and BS(PEG)9. The immobilization yield of RSCR is estimated by protein concentrations of the solution before cross-linking and of the supernatant after spinning down the particles. Cross-linking can be further analyzed by SDS-PAGE and mass spectrometry.

Once RSCR condensates are cross-linked, the capture efficiency of the particles is tested by mixing them with fast protein liquid chromatography (FPLC)-purified SpyTag-GFP solutions with a protein concentration of 2 times molar excess of the initial RSCR solution. After 1 hour of mixing via a tube rotator at 10 rpm, samples are spun down to pellet the RSCR particles and the supernatant is collected for protein concentration measurements as well as SDS-PAGE.

Using a Zeiss LSM900 confocal microscope to perform maximum intensity projection of Z-stack images, the GFP fluorescence of the particles is quantified by image analysis and compared to other particles generated through different cross-linkers. Protein concentrations and fluorescence intensities can be used to estimate capture efficiency. From these results, we can compare RSCR immobilization yields and GFP capture efficiencies to select a cross-linker for further optimization. Possible optimization parameters include cross-linker concentrations, cross-linking temperatures, and cross-linking times. RSCR immobilization yields and GFP capture efficiencies are compared across all cross-linking conditions.

The next step is the capture of SpyTagged enzymes. SpyTagged TAs first from FPLC-purified fractions is captured. Capture of SpyTagged TAs can be verified by measuring the protein concentration of the solution before and after mixing with cross-linked RSCR particles, by labeling enzymes with a maleimide-based fluorescent protein label such as Alexa Fluor 488, BODIPY FL, or FITC, and by performing SDS-PAGE on the enzyme solution. In certain embodiments, TA capture efficiency can also be measured by measuring enzyme concentration before/after using UV absorbance and/or other protein quantitation assays. RSCR::SpyTag-TA particles are spun down, washed, resuspended, and then used for activity assays. Enzyme activity assays can be used to calculate the Michaelis-Menten parameters Kₘ and Vₘₐₓ of immobilized enzymes.

### Capture Efficiency

The capture efficiency is calculated using equation 3:

$$Capture Efficiency ( \% ) = \frac{Amount of Protein Bound to Carrier}{Total Amount of Protein Used} * 100 \% \qquad ( 3 )$$

BCA assays and TA activity assays are performed with at least three technical replicates. Only standard curves with R²≥0.95 and experimental values in the linear range of the assay will be accepted for these assays. Each experiment will be performed three times independently. Statistical significance will be determined using ANOVA and multiple T-tests with Bonferroni correction in Python. The effect of cross-linker length on RSCR immobilization yield and capture efficiency will be examined by calculating either the Pearson's or Spearman's correlation coefficient in Python. Michaelis-Menten parameters will be determined using equation 2.

### Development of Immobilized Biocatalytic Cascades

The cascade reaction of interest is the conversion of benzyl alcohol to benzylamine using an alcohol dehydrogenase (ADH) from Bacillus stearothermophilus (BsADH), an ω-TA from Pseudomonas fluorescens, and an L-alanine dehydrogenase (L-AlaDH) from Bacillus subtilis. ADH converts the benzylalcohol into a hydroxy aldehyde while reducing NAD⁺ to NADH. Then, the ωTA transfers the amine group from L-alanine to the hydroxy aldehyde substrate to result in an amino alcohol and a pyruvate molecule. L-AlaDH acts as an auxiliary enzyme that converts the NADH generated by ADH back to NAD⁺ and recycles the pyruvate generated by ω-TA to L-alanine.¹²⁷ This cascade reaction can be used to convert diols into amino alcohols, which are valuable materials for pharmaceutical manufacturing processes.¹²⁷

We aim to extend the carrier-free and carrier-based immobilization techniques developed herein to co-immobilize ADH, ωTA, and L-AlaDH. For carrier-free immobilization, enzymes can be tagged with an RGG domain to allow them to phase separate. For carrier-based immobilization, enzymes will be tagged with SpyTag003. Fusion enzymes can either be purified by Ni-NTA affinity chromatography or by using a chromatography-free transition temperature cycling protocol.

After purification, fusion enzymes are dialyzed into a Tris-free buffer such as 25 mM HEPES, pH 8 for ADH and L-AlaDH constructs or 25 mM HEPES, 1 mM PLP, pH 8 for ωTA. Protein purity is assessed using SDS-PAGE and protein concentration is measured using the BCA assay. The activity of each enzyme is measured individually. ADH constructs are incubated in a mixture containing 100 mM benzyl alcohol, 1 mM NAD⁺, DMSO 10% in sodium bicarbonate buffer at pH 9 at 30° C. and the increase in absorbance at 340 nm will be monitored by a microplate reader. ωTA construct activity is measured the protocol described above. L-AlaDH constructs is incubated in a 75 mM sodium pyruvate, 500 mM NH₄Cl, 0.5 mM NADH mixture in 100 mM phosphate buffer at pH 8 and 10% DMSO at 25° C., and the decrease in absorbance at 340 nm is monitored by a microplate reader to measure activity. Once all enzymes have been confirmed to be active, then each enzyme is mixed together in a 1:6:10 ADH:ωTA:L-AlaDH activity ratio.¹²⁷ For enzyme-RGG mixtures, half of the solution is phase-separated and cross-linked. For Spy Tagged enzymes, 100 mg of RSCR particles are added to the enzyme solution and incubated for 1 hour at 4° C. The distribution of the immobilized enzyme can be observed by fluorescently labeling enzymes and fluorescent microscopy. The resulting enzyme-RGG particles are washed with buffer 3 times before being resuspended in 25 mM HEPES, 0.35 mM PLP, pH 8 buffer. The immobilization yield will be analyzed by performing SDS-PAGE on samples of the ADH:ωTA:L-AlaDH solutions along with protein quantitation assays before and after immobilization. To measure activity of free enzymes and immobilized particles, the enzyme mixtures will be added to solutions containing 50 mM benzyl alcohol, 0.75 mM NAD⁺, 0.35 mM PLP, 250 mM L-alanine, 275 mM NH₄Cl, 10% DMSO, pH 8.5 and incubated at 25° C. in a tube rotator at 40 rpm. Reactions can be monitored by a microplate reader using UV absorbance in tandem with high performance liquid chromatography (HPLC). Liquid chromatography-mass spectrometry (LC-MS) could also be used to measure reaction progress. After the conclusion of a reaction, the immobilized enzymes are spun down, resuspended with 25 mM HEPES, 0.1 mM PLP, pH 8.5 buffer, and used for another reaction cycle to observe the operational stability.

Individual enzyme activities are compared to a no-enzyme control using ANOVA and multiple T-tests with Bonferroni correction to confirm enzyme activity. The reaction yield of multiple co-immobilized enzymes is compared to free enzyme solutions using paired T-tests. The localization and distribution of immobilized fluorescently labeled enzymes is measured by image processing either using the Zeiss ZEN microscopy software or using open-source packages such as FIJI. Residual activity is then plotted using Python.

The following examples are provided to facilitate the practice of the invention. They are not intended to limit the invention in any way.

### Example 1

### Validating the Preservation of Enzymatic Activity in Cross-Linked Enzyme-RGG Condensates

The retention of enzyme activity after immobilization is paramount for an immobilized enzyme's utility in catalytic processes. Therefore, this experiment demonstrates that enzyme biomolecular condensates remain active after cross-linking.

Enzyme candidates for immobilization are fused to the intrinsically disordered RGG protein, which is derived from the N-terminal disordered domain of the C. elegans LAF-1 protein. After inducing phase separation, enzyme condensates are cross-linked to immobilize them. The data described herein indicates that enzymes remain active after cross-linking and operate in a wider range of conditions than soluble enzymes. Our approach analyzes immobilization yield using protein quantification assays and sodium dodecyl sulfate polyacrylamide gel electrophoresis (SDS-PAGE). Then, we use enzyme activity assays to measure residual activity after immobilization. The expected outcome of this experiment is the identification of enzyme-RGG fusion proteins that will remain active after phase separation and cross-linking.

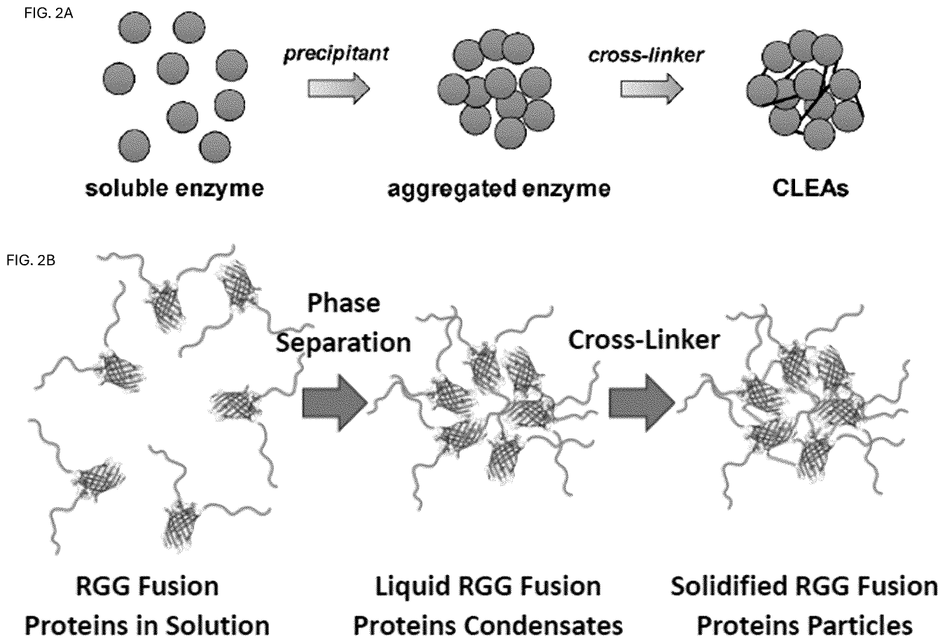

In previously published methods, enzymes are concentrated either by crystallization (in the case of cross-linked enzyme crystals (CLECs)) or by aggregation (in the case of cross-linked enzyme aggregates (CLEAs)).^{45,95} Enzymes in close proximity can subsequently be cross-linked to immobilize the enzyme and stabilize their structures (FIG. 2A). In the presently described method, leveraging the self-assembling properties of the intrinsically disordered RGG protein allows for the concentration of enzymes into condensates for cross-linking (FIG. 2B). The RGG protein has been demonstrated to be sufficient for phase separation and can be used as a protein tag to allow other folded proteins to form biomolecular condensates.^{81,96} In CLEA formation, precipitants such as ammonium sulfate, acetone, and ethanol can be used to aggregate enzymes, but these can compromise the enzymatic structure and lead to a loss of activity.^{51,97} In contrast, RGG phase separates at low temperatures and in low saline concentrations, and these mild conditions are ideal for preserving the enzyme's active form.⁹⁶

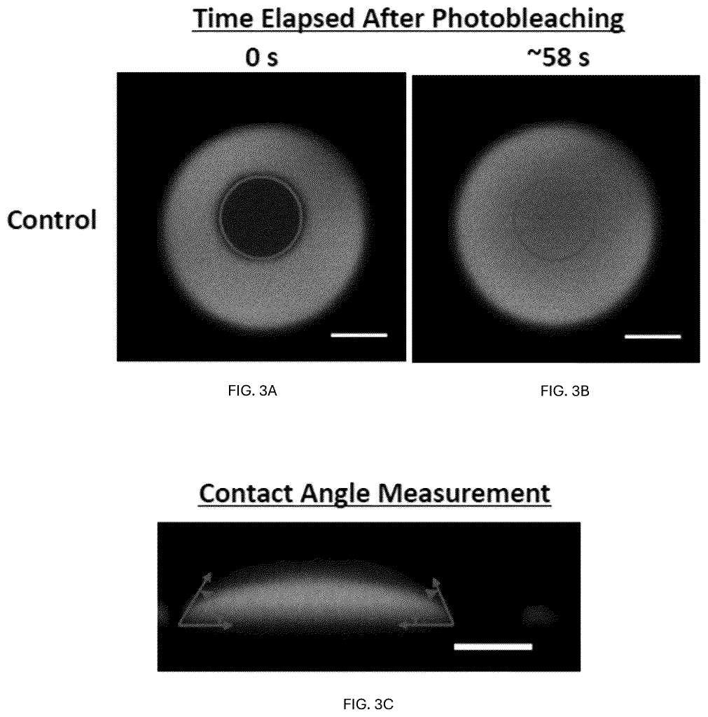

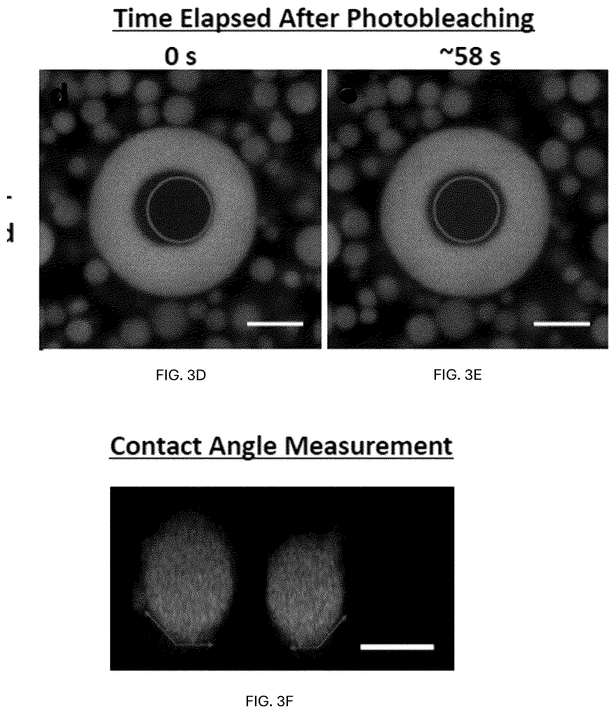

To test the feasibility of cross-linking biomolecular condensates, GFP-RGG fusion proteins (GFP-RGG-RGG and RGG-GFP-RGG) were used as a model system. After incubating GFP-RGG samples on ice to induce phase separation and cross-linking the primary amine-reactive cross-linker bissulfosuccinimidyl suberate (BS3) in 20 times molar excess of the protein sample for 2 hours on ice, the material properties of these condensates were probed using a Zeiss LSM900 confocal microscope and the cross-linked condensate samples were qualitatively analyzed through SDS-PAGE. Compared to untreated GFP-RGG condensates, cross-linked condensates do not recover fluorescence after photobleaching (FIG. 3B,3E) due to a lack of diffusion occurring. Additionally, cross-linked condensates do not wet untreated glass surfaces like GFP-RGG condensates normally do (FIG. 3C,3F).

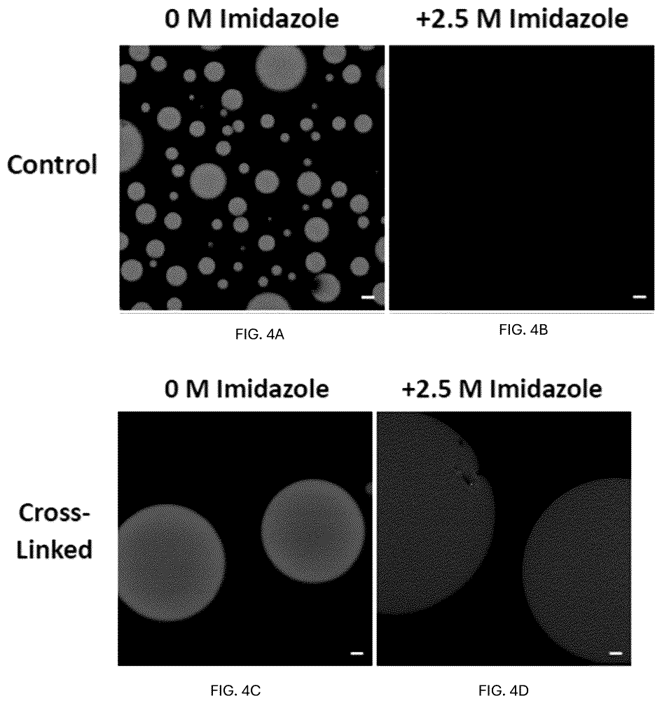

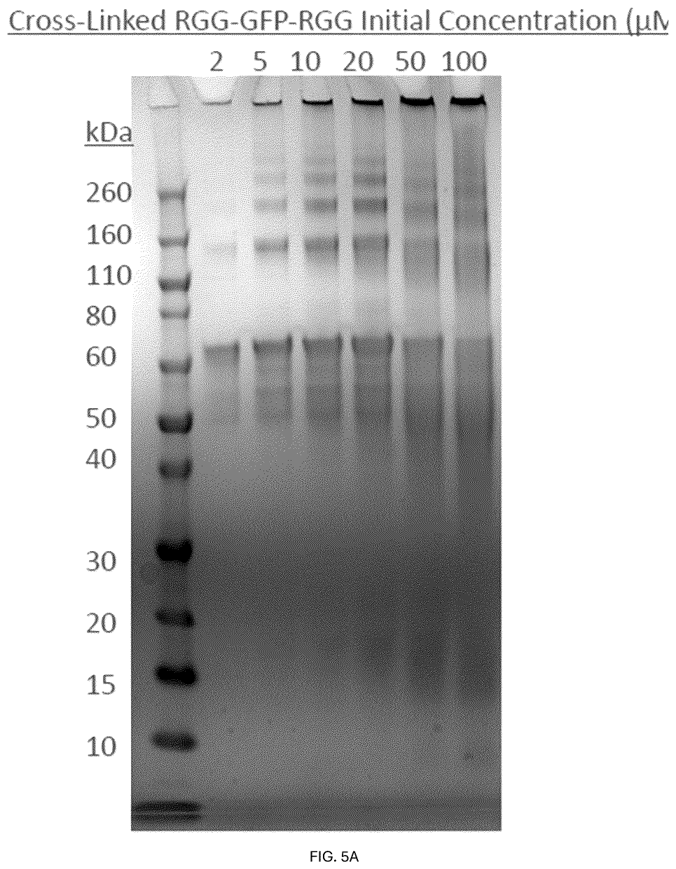

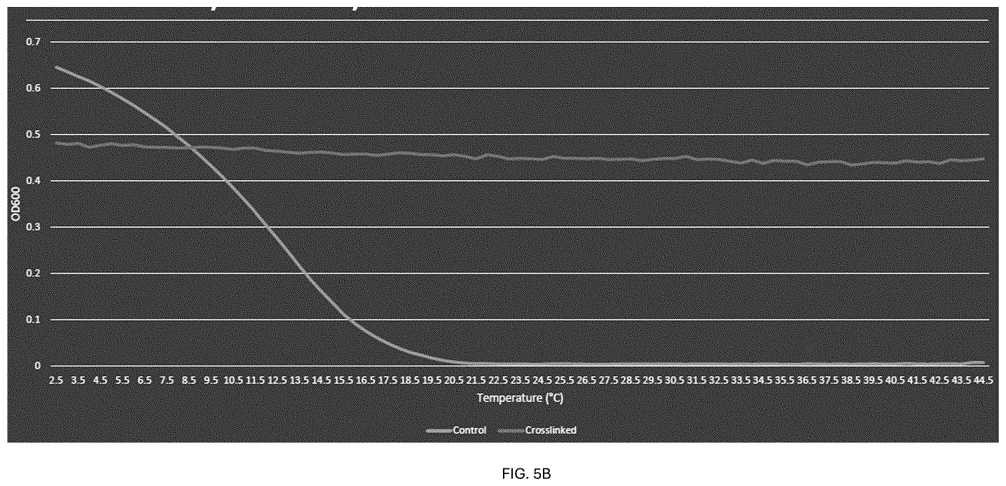

Cross-linked GFP-RGG condensates can also tolerate harsher conditions than non-cross-linked condensates such as high imidazole concentrations (FIG. 4). When cross-linked samples are run through an SDS-PAGE gel, a large portion of the sample does not migrate through the gel and multimeric bands appear (FIG. 5A). The cross-linking was also validated by turbidity assay (FIG. 5B). The shift in properties after cross-linking provides evidence that biomolecular condensates can be cross-linked to form the basis of solid materials.

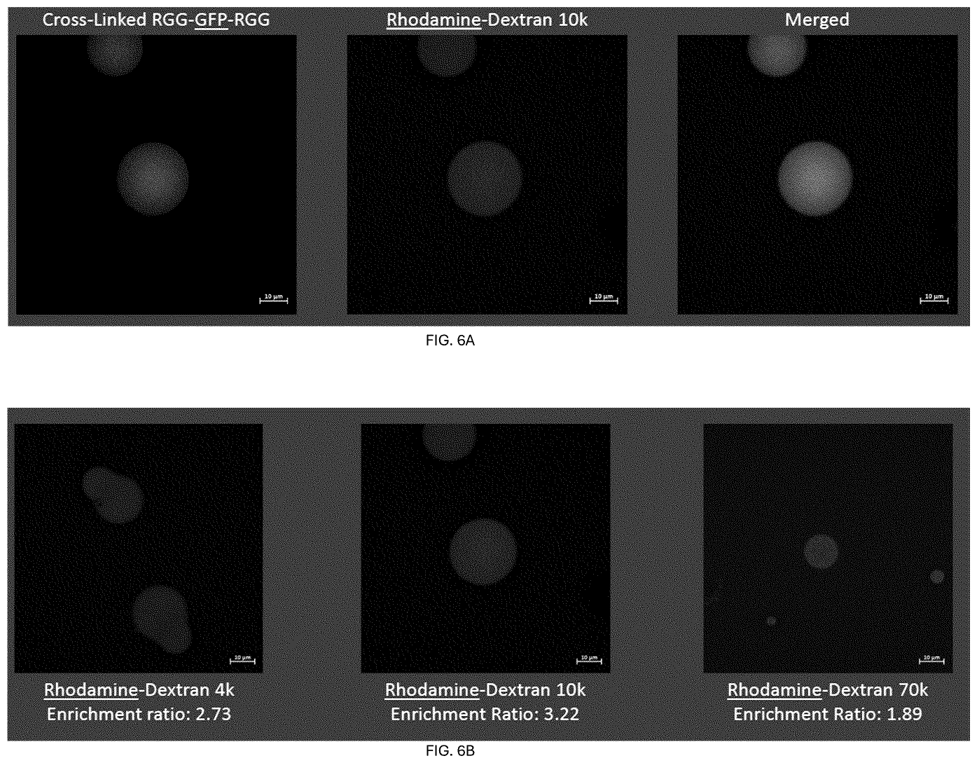

Next, we tested whether the cross-linked condensates are permeable to small molecules and are capable of enriching target molecules. Condensate permeability is useful in the described condensates as this property allows the condensate to circumvent mass transfer limitations encountered with other immobilization techniques. Permeability allows molecules to enter the interior of the particle which allows for higher catalytic productivity by increasing enzyme loading and reducing diffusional limitations. Having uniform enzyme distribution throughout the particle further increases the enzyme accessibility to substrates and reduces activity dilution. To support the permeability of the condensates, we used rhodamine-labeled dextrans of varying molecular sizes to characterize the permeability of crosslinked condensates through fluorescent microscopy. For dextrans of all sizes, the rhodamine fluorescence intensity is higher than the surrounding solution. This indicates that the condensates are permeable to other molecules and can enrich them, which would be useful to reduce diffusional limitations of immobilized enzymes and potentially increase enzyme reaction rates (See FIG. 6A-6B).

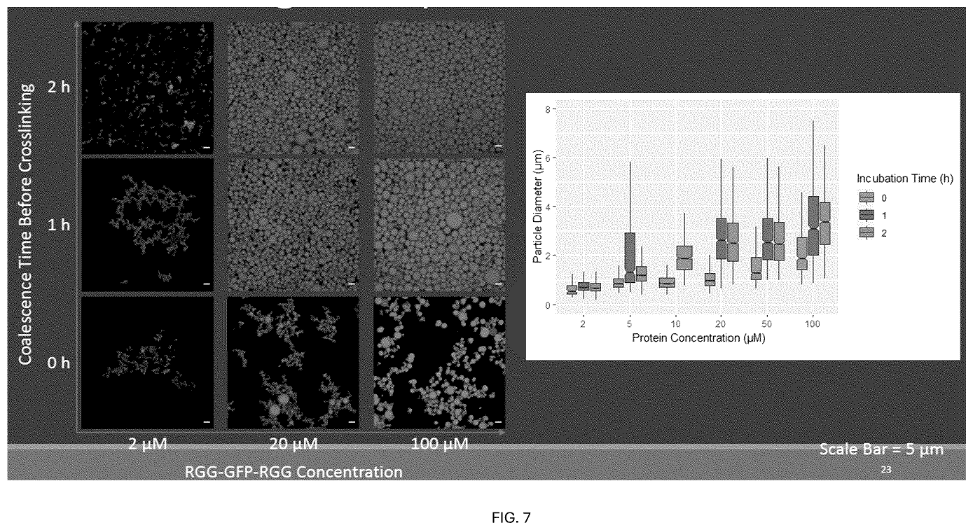

RGG fusion proteins have been successfully cross-linked using the primary amine-reactive crosslinker BS3. By modifying cross-linking conditions, the size of resulting particles can be controlled and under the conditions tested. In the present experiment, median particle diameters ranged from 0.55 μm to 3.38 μm. If the condensate is allowed to coalesce and/or the protein concentration is increased before crosslinking, a biomolecular condensate with a larger particle size after crosslinking can be formed. (FIG. 7). Because of the general similarities between current carrier-free immobilization techniques and the proposed phase-separation method, enzymes also remain active after cross-linking. In addition, phase separation can provide a more broadly applicable method to enzyme sequestration, abrogating the need to screen precipitants as is typically performed during CLEA formation.

Previous PfωTA through CLEA formation resulted in CLEAs that retained 29% of their initial activity and achieved maximum thermostability at 60-70° C.⁵¹ The method of cross-linking TA-RGG condensates disclosed herein will result in immobilized TAs with increased activity and thermostability when compared to previous methods. The TAs, information derived from TA activity assays, and cross-linking conditions are carried over to carrier-based immobilization methods in Example 2.

### Example 2

### Demonstrating the Utility of Cross-Linked RGG Condensates as a Carrier Material for Capturing and Immobilizing Free Enzymes

Carrier-free immobilization can be used over a broad range of enzymes. In this experiment, we utilize cross-linked RGG as a carrier material for capturing and immobilizing free enzymes from solution. The catalytically active enzymes will be captured into a biomolecular condensate by encoding specific protein-protein interactions into intrinsically disordered proteins (IDPs).

Forming biomolecular condensates from IDPs is possible due to multivalent interactions. In cells, some IDPs can serve as scaffolds that recruit client molecules into biomolecular condensates through multivalent interactions.^{69,79} For example, in C. elegans, the scaffold protein SPD-5 can self-assemble into condensates and recruit the microtubule polymerase ZYG-9 and the microtubule-stabilizing protein TPXL-1, promoting microtubule aster nucleation.⁷¹ Drawing inspiration from these biologically-occurring phenomena, researchers have engineered IDPs with specific protein-protein interactions encoded into the amino acid sequence. By doing so, cargo proteins tagged with a corresponding domain can be recruited into biomolecular condensates.^{89,96}

The experimental approach utilizes cross-linked SpyCatcher-RGG proteins that conjugate specifically with a SpyTagged enzyme. This method simplifies immobilization workflows and provides a modular platform where carrier materials and enzymes can be optimized independently.

The SpyTag/SpyCatcher system allows for the irreversible conjugation of recombinant proteins through the formation of an intermolecular isopeptide bond between the SpyTag peptide and SpyCatcher protein. The system was developed by splitting and engineering the CnaB2 domain of the FbaB adhesin protein from Streptococcus pyogenes.¹⁰⁷ Spy Tagged proteins react with SpyCatcher in high specificity with a second-order rate constant of 1.4×10³ M⁻¹ s⁻¹.¹⁰⁷ The primary outcome of this experiment is development of a novel protein-based carrier material for enzyme immobilization.

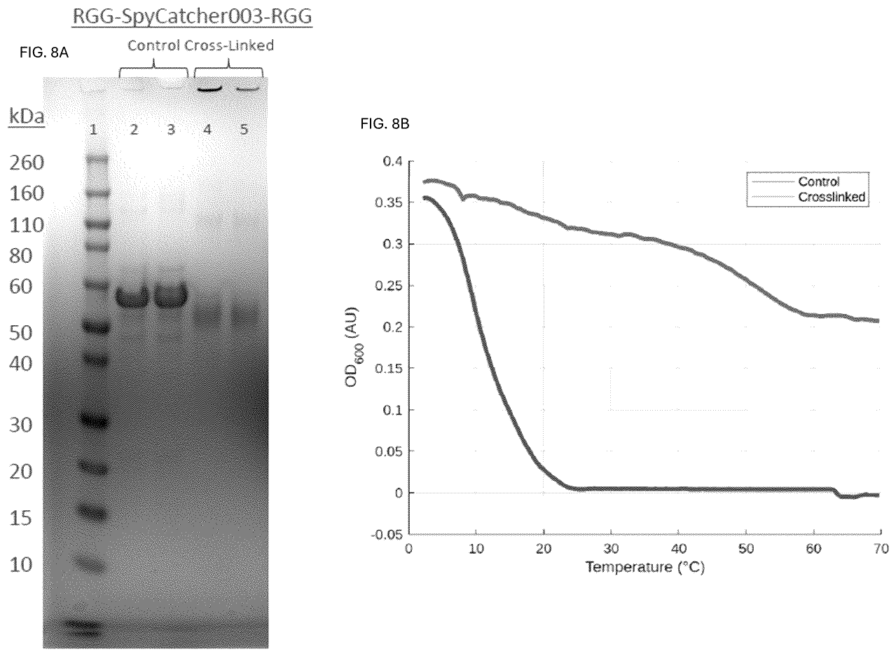

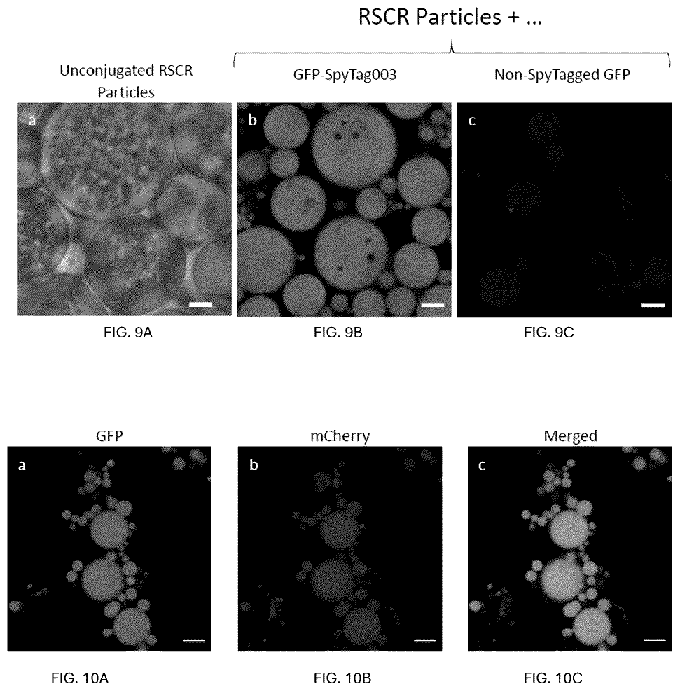

RGG-SpyCatcher003-RGG (RSCR), SpyTagged-GFP (SpyTag003-GFP and GFP-SpyTag003), and (as a control) a non-SpyTagged GFP (SYNZIP2-GFP) were cloned, expressed in BL21(DE3) E. coli, purified by Ni-NTA affinity chromatography, and dialyzed into 150 mM NaCl, 50 mM borate, pH 8.0 buffer. Phase separation was induced in RSCR samples by incubating on ice for 1 hour, and the condensates were cross-linked with BS3 in 20 times molar excess for 2 hours on ice. The cross-linking was validated by SDS-PAGE and turbidity assays (FIG. 8). The cross-linked RSCR particles were spun down and washed three times using 150 mM NaCl, 50 mM borate, pH 8.0 buffer. Then, RSCR particles were mixed with SpyTagged GFP samples in equimolar ratios using a tube rotator at 10 rpm for 1 hour, after which the particles were spun down, the supernatant was collected, and RSCR particles were washed three times using 150 mM NaCl, 50 mM borate, pH 8.0 buffer. Fluorescence microscopy revealed that the RSCR particles mixed with SpyTagged GFP solutions (FIG. 9B) had a higher fluorescence signal than particles mixed with non-SpyTagged GFP (FIG. 9C), indicating that the SpyCatcher domain is capable of capturing Spy Tagged proteins even after cross-linking. In addition, GFP fluorescence was distributed throughout the RSCR particles (FIG. 9B), indicating that RSCR particles are porous enough to allow for GFP to diffuse and be captured in the interior of the particle.

These results have demonstrated that SpyCatcher domains remain active after crosslinking and are able to capture SpyTagged GFP from solution. Other researchers have also reported the use of the SpyTag/SpyCatcher system for enzyme immobilization. Therefore, it stands that cross-linked SpyCatcher-RGG particles could be used to capture and immobilize enzymes. Our proposed method mirrors previously established enzyme immobilization techniques and provides several advantages. Enzymes can be covalently bound to support materials by cross-linking amino acids on the enzyme surface to the carrier, providing a strong bond that prevents enzyme leaching into solution. However, if the support material is not reactive with the cross-linker of choice, then the carrier material must be functionalized.

In the system disclosed herein, the molecular interaction is encoded into the SpyCatcher domain, which would negate the need for additional functionalization steps. Our preliminary research indicates that cross-linked SpyCatcher-RGG particles are porous enough that SpyTagged GFP can diffuse into the particle and be captured in the interior. When translated to enzyme immobilization contexts, this property leads to higher amounts of enzyme loading. Furthermore, the nanopores prevent microbes from entering the particle and causing contamination. Since the SpyTag/SpyCatcher system is highly bioorthogonal, cross-linked SpyCatcher-RGG particles could be used to capture SpyTagged enzymes from lysate, providing a 1-step method for purification and immobilization. Enzyme denaturation and structural distortion during immobilization can be minimized because the SpyTag on the enzyme provides a “tether” for covalent binding instead of utilizing surface amino acids.^{33,113}

For all amine-reactive cross-linkers, prior data suggests a reduction in capture efficiency will be observed compared to soluble RSCR because SpyCatcher relies on a lysine residue to form an isopeptide bond with SpyTag.¹⁰⁷ Additionally, the data suggests that longer cross-linkers will lead to higher recovered capture efficiency since the longer cross-linkers are less likely to fit into the active site of SpyCatcher and react with the catalytic lysine residue. Based on these results, the data suggests that SpyTagged TAs will be captured into RSCR particles. The data also indicates that the captured TAs will remain active since there should be a reduced chance of enzyme structure distortion.

### Example 3

Development of Immobilized Biocatalytic Cascades by Co-Immobilizing Multiple Enzymes into Cross-Linked RGG Condensates

Multiple enzymes can be immobilized to form a coordinated biocatalytic cascade that can efficiently execute multistep enzymatic reactions. Here, we extend the enzyme immobilization techniques described above to immobilize multiple enzymes and form an immobilized biocatalytic cascade. Both our carrier-free approach from Example 1 using RGG-enzyme fusions and carrier-based approaches from Example 2 using SpyTag/SpyCatcher conjugation are used to immobilize enzyme cascades. Furthermore, we immobilize enzyme cascades that involve TAS, focusing specifically on an enzyme cascade that converts alcohols to amino alcohols using alcohol dehydrogenases, w-transaminases, and L-alanine dehydrogenases. By simplifying enzyme co-immobilization using cross-linked biomolecular condensates, we increase residual enzyme activity after immobilization. The co-immobilized enzymes will remain active and our novel methods of immobilization will result in higher reaction yields than other methods used in previously published studies.

Cascade reactions offer many advantages over classical multi-step reactions such as simplified reaction schemes and the potential for higher yields.¹¹⁸ Enzymes are particularly adept for performing cascade reactions since they are generally active in aqueous buffers, similar pHs, and similar temperatures.¹¹⁹ By co-immobilizing enzymes, the reaction rate of a reaction can be accelerated because the distance that an intermediate from one enzyme needs to travel to reach the second enzyme is reduced.¹²⁰

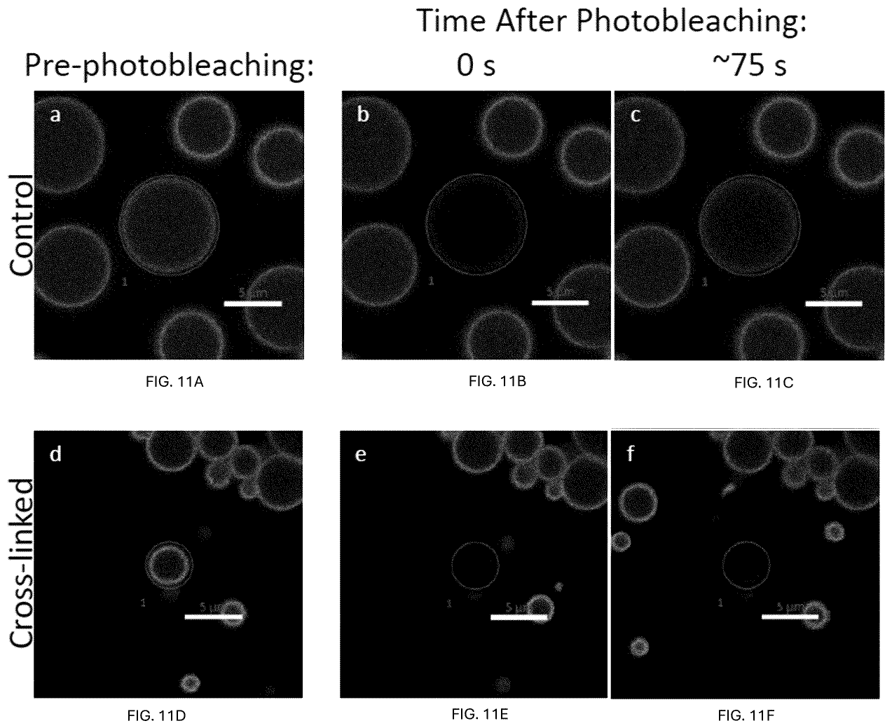

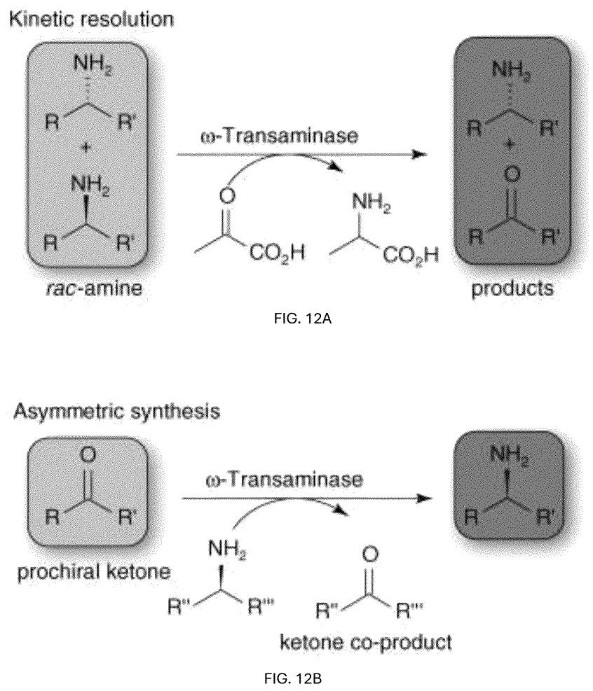

Different fusion proteins tagged with RGG domains have been found to co-localize into the same biomolecular condensates owing to the RGG domain's self-assembling properties. For example, mixtures of RGG-GFP-RGG and RGG-mCherry-RGG will co-phase separate to form condensates containing both components (FIG. 10). We formed core-shell condensates by mixing the phase separating RGG-RGG protein with non-phase separating, surfactant-like protein constructs such as MBP-GFP-RGG.¹²⁶ These core-shell condensates have been cross-linked using BS3 to generate core-shell particles (FIG. 11).

Many published articles have reported successful co-immobilization of transaminase-containing enzyme cascades through both carrier-based and carrier-free methods. Similarly, our novel immobilization techniques utilizing cross-linked biomolecular condensates is applicable to enzymes other than TAs. By co-immobilizing enzymes using our novel technique, not only do we demonstrate the broad applicability of our method over a wider range of enzymes, but we also show that it can be used to facilitate enzyme cascade immobilization. RGG-tagged proteins have been shown to co-phase separate, which facilitates the colocalization and cross-linking of multiple enzymes as part of a carrier-free approach. Cross-linked RGG-SpyCatcher-RGG particles could be used as a carrier material to capture multiple enzymes from a mixture.

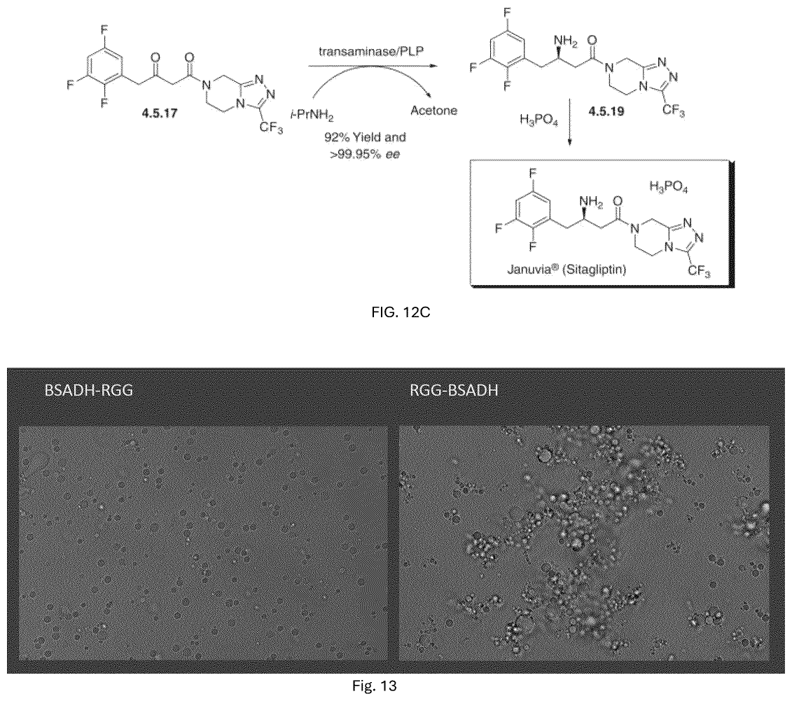

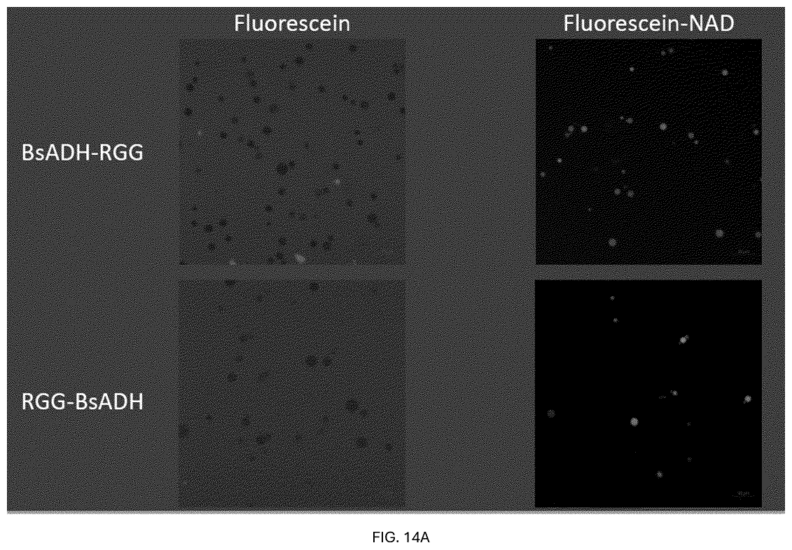

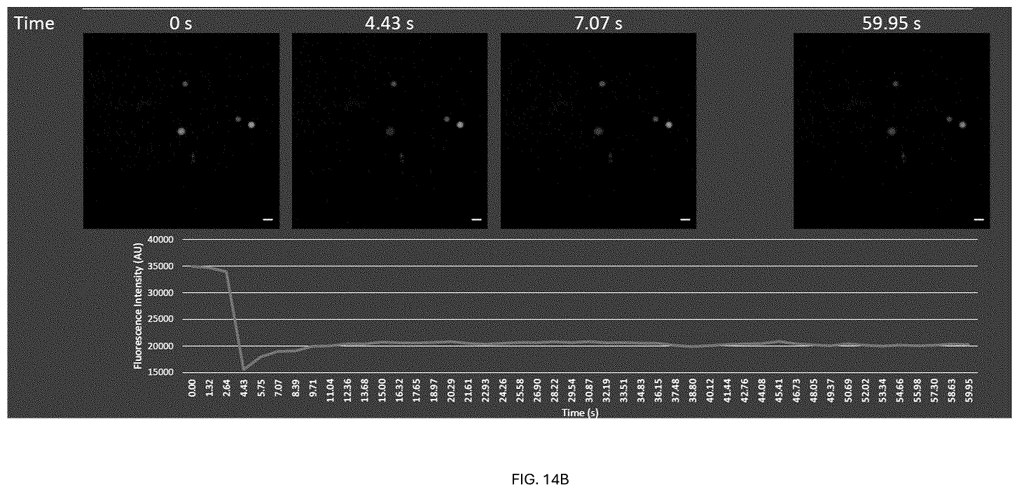

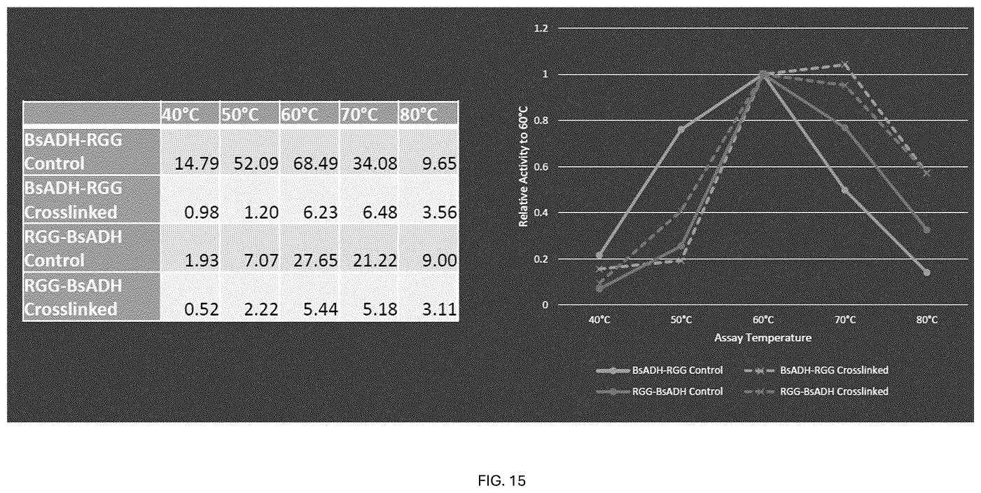

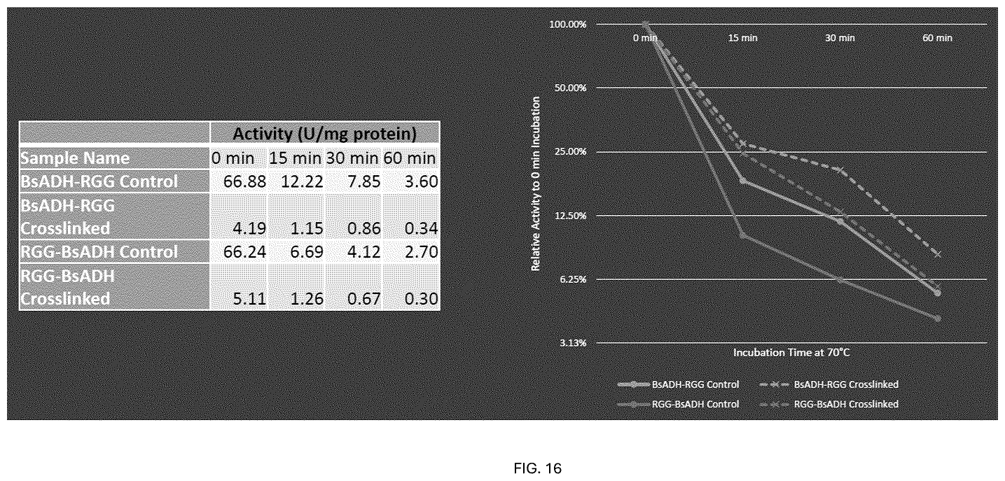

With our novel immobilization methods, ADH, ωTA, and L-AlaDH can be co-immobilized to form an active biocatalytic cascade. Exemplary images of RGG-BsADH show that this fusion protein forms cross-linked enzyme condensates. (FIG. 13) Using fluorescence imaging, we were also able to show that NAD⁺ permeates the cross-linked BsADH condensates (FIG. 14A-14B). Next, we analyzed whether the cross-linked BsADH condensates retained activity at different temperatures. In both the BsADH-RGG and RGG-BsADH cross-linked condensates, the proteins retained more activity at higher temperatures than the controls (Figure. 15). Next, we analyzed the activity of the BsADH condensates after incubation at 70° C. over 60 minutes. Throughout the 60 minutes, both BsADH cross-linked condensates retained more activity than the controls (FIG. 16).

Unlike currently used techniques, the enzymes described herein can be reused for multiple cycles. Our enzyme are also evenly distributed throughout the cross-linked condensate in both carrier-based and carrier-free immobilization methods. This even distribution increases enzyme accessibility to substrates and reduces activity dilution.

### REFERENCES

(1) Operando Studies in Heterogeneous Catalysis; Springer Berlin Heidelberg: New York, NY, 2016.
(2) Hart, A. Circular Economy: Closing the Catalyst Loop with Metal Reclamation from Spent Catalysts, Industrial Waste, Waste Shells and Animal Bones. Biomass Conv. Bioref. 2023, 13 (13), 11483-11498. https://doi.org/10.1007/s13399-021-01942-8.
(3) Schlögl, R. Heterogeneous Catalysis. Angewandte Chemie International Edition 2015, 54 (11), 3465-3520. https://doi.org/10.1002/anie.201410738.
(4) Bai, S.-T.; Smet, G. D.; Liao, Y.; Sun, R.; Zhou, C.; Beller, M.; W. Maes, B. U.; F. Sels, B. Homogeneous and Heterogeneous Catalysts for Hydrogenation of CO 2 to Methanol under Mild Conditions. Chemical Society Reviews 2021, 50 (7), 4259-4298. https://doi.org/10.1039/D0CS01331E.
(5) Marocco Stuardi, F.; MacPherson, F.; Leclaire, J. Integrated CO2 Capture and Utilization: A Priority Research Direction. Current Opinion in Green and Sustainable Chemistry 2019, 16, 71-76. https://doi.org/10.1016/j.cogsc.2019.02.003.
(6) Alberico, E.; Nielsen, M. Towards a Methanol Economy Based on Homogeneous Catalysis: Methanol to H2 and CO2 to Methanol. Chem. Commun. 2015, 51 (31), 6714-6725. https://doi.org/10.1039/C4CC09471A.
(7) Navarro-Jaen, S.; Virginie, M.; Bonin, J.; Robert, M.; Wojcieszak, R.; Khodakov, A. Y. Highlights and Challenges in the Selective Reduction of Carbon Dioxide to Methanol. Nat Rev Chem 2021, 5 (8), 564-579. https://doi.org/10.1038/s41570-021-00289-y.
(8) Dai, Y.; Xiong, Y. Control of Selectivity in Organic Synthesis via Heterogeneous Photocatalysis under Visible Light. Nano Research Energy 2022, 1 (1), e9120006. https://doi.org/10.26599/NRE.2022.9120006.
(9) Metzger, E. D.; Comito, R. J.; Wu, Z.; Zhang, G.; Dubey, R. C.; Xu, W.; Miller, J. T.; Dincǎ, M. Highly Selective Heterogeneous Ethylene Dimerization with a Scalable and Chemically Robust MOF Catalyst. ACS Sustainable Chem. Eng. 2019, 7 (7), 6654-6661. https://doi.org/10.1021/acssuschemeng.8b05703.
(10) Yang, X.-F.; Wang, A.; Qiao, B.; Li, J.; Liu, J.; Zhang, T. Single-Atom Catalysts: A New Frontier in Heterogeneous Catalysis. Acc. Chem. Res. 2013, 46 (8), 1740-1748. https://doi.org/10.1021/ar300361m.
(11) Védrine, J. C. Metal Oxides in Heterogeneous Oxidation Catalysis: State of the Art and Challenges for a More Sustainable World. ChemSusChem 2019, 12 (3), 577-588. https://doi.org/10.1002/cssc.201802248.
(12) Xia, Y.; Yang, X. Toward Cost-Effective and Sustainable Use of Precious Metals in Heterogeneous Catalysts. Acc. Chem. Res. 2017, 50 (3), 450-454. https://doi.org/10.1021/acs.accounts.6b00469.
(13) Thomas, J. M.; Thomas, W. J. Principles and Practice of Heterogeneous Catalysis, Second, revised edition.; Wiley-VCH, Verlag GmbH & Co. KGaA: Weinheim, 2015.
(14) Tayebi-Khorami, M.; Edraki, M.; Corder, G.; Golev, A. Re-Thinking Mining Waste through an Integrative Approach Led by Circular Economy Aspirations. Minerals 2019, 9 (5), 286. https://doi.org/10.3390/min9050286.
(15) Israf Ud Din; Nasir, Q.; Garba, M. D.; Alharthi, A. I.; Alotaibi, M. A.; Usman, M. A Review of Preparation Methods for Heterogeneous Catalysts. Mini-Reviews in Organic Chemistry 2022, 19 (1), 92-110. https://doi.org/10.2174/1570193X18666210308151136.
(16) Campanati, M.; Fornasari, G.; Vaccari, A. Fundamentals in the Preparation of Heterogeneous Catalysts. Catalysis Today 2003, 77 (4), 299-314. https://doi.org/10.1016/S0920-5861(02)00375-9.
(17) Delmon, B. Preparation of Heterogeneous Catalysts. J Therm Anal Calorim 2007, 90 (1), 49-65. https://doi.org/10.1007/s10973-007-8476-y.
(18) Srinivasan, B. A Guide to the Michaelis-Menten Equation: Steady State and Beyond. The FEBS Journal 2022, 289 (20), 6086-6098. https://doi.org/10.1111/febs.16124.
(19) Industrial Biotransformations, 2nd completely rev. and extended ed.; Liese, A., Seelbach, K., Wandrey, C., Eds.; Wiley-VCH: Weinheim, 2006.
(20) A. Sheldon, R.; Pelt, S. van. Enzyme Immobilisation in Biocatalysis: Why, What and How. Chemical Society Reviews 2013, 42 (15), 6223-6235. https://doi.org/10.1039/C3CS60075K.
(21) Kim, S. M.; Lee, J.; Kang, S. H.; Heo, Y.; Yoon, H.-J.; Hahn, J.-S.; Lee, H. H.; Kim, Y. H. O2-Tolerant CO Dehydrogenase via Tunnel Redesign for the Removal of CO from Industrial Flue Gas. Nat Catal 2022, 5 (9), 807-817. https://doi.org/10.1038/s41929-022-00834-y.
(22) Jørgensen, H.; Pinelo, M. Enzyme Recycling in Lignocellulosic Biorefineries. Biofuels, Bioproducts and Biorefining 2017, 11 (1), 150-167. https://doi.org/10.1002/bbb.1724.
(23) Gherardi, F.; Turyanska, L.; Ferrari, E.; Weston, N.; Fay, M. W.; Colston, B. J. Immobilized Enzymes on Gold Nanoparticles: From Enhanced Stability to Cleaning of Heritage Textiles. ACS Appl. Bio Mater. 2019, 2 (11), 5136-5143. https://doi.org/10.1021/acsabm.9b00802.
(24) Sohrabi, N.; Rasouli, N.; Torkzadeh, M. Enhanced Stability and Catalytic Activity of Immobilized α-Amylase on Modified Fe3O4 Nanoparticles. Chemical Engineering Journal 2014, 240, 426-433. https://doi.org/10.1016/j.cej.2013.11.059.
(25) Rodrigues, R. C.; Ortiz, C.; Berenguer-Murcia, Ã.; Torres, R.; Fernández-Lafuente, R. Modifying Enzyme Activity and Selectivity by Immobilization. Chem. Soc. Rev. 2013, 42 (15), 6290-6307.https://doi.org/10.1039/C2CS35231A.
(26) Lei, C.; Soares, T. A.; Shin, Y.; Liu, J.; Ackerman, E. J. Enzyme Specific Activity in Functionalized Nanoporous Supports. Nanotechnology 2008, 19 (12), 125102. https://doi.org/10.1088/0957-4484/19/12/125102.
(27) Guzik, U.; Hupert-Kocurek, K.; Wojcieszyńska, D. Immobilization as a Strategy for Improving Enzyme Properties-Application to Oxidoreductases. Molecules 2014, 19 (7), 8995-9018. https://doi.org/10.3390/molecules19078995.
(28) Zdarta, J.; Jankowska, K.; Bachosz, K.; Degórska, O.; Kaźmierczak, K.; Nguyen, L. N.; Nghiem, L. D.; Jesionowski, T. Enhanced Wastewater Treatment by Immobilized Enzymes. Curr Pollution Rep 2021, 7 (2), 167-179. https://doi.org/10.1007/s40726-021-00183-7.
(29) Jun, L. Y.; Yon, L. S.; Mubarak, N. M.; Bing, C. H.; Pan, S.; Danquah, M. K.; Abdullah, E. C.; Khalid, M. An Overview of Immobilized Enzyme Technologies for Dye and Phenolic Removal from Wastewater. Journal of Environmental Chemical Engineering 2019, 7 (2), 102961. https://doi.org/10.1016/j.jece.2019.102961.
(30) Dekker, P. J. T.; Koenders, D.; Bruins, M. J. Lactose-Free Dairy Products: Market Developments, Production, Nutrition and Health Benefits. Nutrients 2019, 11 (3), 551. https://doi.org/10.3390/nu11030551.
(31) Rajdeo, K.; Harini, T.; Lavanya, K.; Fadnavis, N. W. Immobilization of Pectinase on Reusable Polymer Support for Clarification of Apple Juice. Food and Bioproducts Processing 2016, 99, 12-19. https://doi.org/10.1016/j.fbp.2016.03.004.
(32) Nelson, J. M.; Griffin, E. G. ADSORPTION OF INVERTASE. J. Am. Chem. Soc. 1916, 38 (5), 1109-1115. https://doi.org/10.1021/ja02262a018.
(33) Zucca, P.; Sanjust, E. Inorganic Materials as Supports for Covalent Enzyme Immobilization: Methods and Mechanisms. Molecules 2014, 19 (9), 14139-14194. https://doi.org/10.3390/molecules190914139.
(34) de Souza Lima, J.; Boemo, A. P. S. I.; de Araújo, P. H. H.; de Oliveira, D. Immobilization of Endoglucanase on Kaolin by Adsorption and Covalent Bonding. Bioprocess Biosyst Eng 2021, 44 (8), 1627-1637. https://doi.org/10.1007/s00449-021-02545-3.
(35) Dey, G.; Nagpal, V.; Banerjee, R. Immobilization of α-Amylase from Bacillus Circulans GRS 313 on Coconut Fiber. Appl Biochem Biotechnol 2002, 102 (1), 303-313. https://doi.org/10.1385/ABAB: 102-103:1-6:303.
(36) Sharma, S.; Kaur, P.; Jain, A.; Rajeswari, M. R.; Gupta, M. N. A Smart Bioconjugate of Chymotrypsin. Biomacromolecules 2003, 4 (2), 330-336. https://doi.org/10.1021/bm0256799.
(37) Petkova, G. A.; Záruba, K.; Král, V. Synthesis of Silica Particles and Their Application as Supports for Alcohol Dehydrogenases and Cofactor Immobilizations: Conformational Changes That Lead to Switch in Enzyme Stereoselectivity. Biochimica et Biophysica Acta (BBA)-Proteins and Proteomics 2012, 1824 (6), 792-801. https://doi.org/10.1016/j.bbapap.2012.03.010.
(38) Nguyen, H. H.; Kim, M. An Overview of Techniques in Enzyme Immobilization. Applied Science and Convergence Technology 2017, 26 (6), 157-163. https://doi.org/10.5757/ASCT.2017.26.6.157.
(39) Tepe, O.; Dursun, A. Y. Combined Effects of External Mass Transfer and Biodegradation Rates on Removal of Phenol by Immobilized Ralstonia Eutropha in a Packed Bed Reactor. J Hazard Mater 2008, 151 (1), 9-16. https://doi.org/10.1016/j.jhazmat.2007.05.049.
(40) Sonwani, R. K.; Giri, B. S.; Das, T.; Singh, R. S.; Rai, B. N. Biodegradation of Fluorene by Neoteric LDPE Immobilized Pseudomonas Pseudoalcaligenes NRSS3 in a Packed Bed Bioreactor and Analysis of External Mass Transfer Correlation. Process Biochemistry 2019, 77, 106-112. https://doi.org/10.1016/j.procbio.2018.11.015.
(41) Swain, G.; Sonwani, R. K.; Giri, B. S.; Singh, R. S.; Jaiswal, R. P.; Rai, B. N. A Study of External Mass Transfer Effect on Biodegradation of Phenol Using Low-Density Polyethylene Immobilized Bacillus flexus GS1 IIT (BHU) in a Packed Bed Bioreactor. Water and Environment Journal 2021, 35 (1), 285-294. https://doi.org/10.1111/wej.12626.
(42) Dursun, A. Y.; Tepe, O. Internal Mass Transfer Effect on Biodegradation of Phenol by Ca-Alginate Immobilized Ralstonia Eutropha. J Hazard Mater 2005, 126 (1-3), 105-111. https://doi.org/10.1016/j.jhazmat.2005.06.013.
(43) Xu, B.; Azam, S. M. R.; Feng, M.; Wu, B.; Yan, W.; Zhou, C.; Ma, H. Application of Multi-Frequency Power Ultrasound in Selected Food Processing Using Large-Scale Reactors: A Review. Ultrason Sonochem 2021, 81, 105855. https://doi.org/10.1016/j.ultsonch.2021.105855.
(44) Guisan, J. M.; López-Gallego, F.; Bolivar, J. M.; Rocha-Martin, J.; Fernandez-Lorente, G. The Science of Enzyme Immobilization. In Immobilization of Enzymes and Cells: Methods and Protocols; Guisan, J. M., Bolivar, J. M., López-Gallego, F., Rocha-Martin, J., Eds.; Methods in Molecular Biology; Springer US: New York, NY, 2020; pp 1-26. https://doi.org/10.1007/978-1-0716-0215-7_1.
(45) Quiocho, F. A.; Richards, F. M. Intermolecular Cross Linking of a Protein in the Crystalline State: Carboxypeptidase-A*. Proceedings of the National Academy of Sciences 1964, 52 (3), 833-839. https://doi.org/10.1073/pnas.52.3.833.
(46) Zelinski, T.; Waldmann, H. Cross-Linked Enzyme Crystals (CLECs): Efficient and Stable Biocatalysts for Preparative Organic Chemistry. Angewandte Chemie International Edition in English 1997, 36 (7), 722-724. https://doi.org/10.1002/anie.199707221.
(47) Goetze, D.; Foletto, E. F.; da Silva, H. B.; Silveira, V. C. C.; Dal Magro, L.; Rodrigues, R. C. Effect of Feather Meal as Proteic Feeder on Combi-CLEAs Preparation for Grape Juice Clarification. Process Biochemistry 2017, 62, 122-127. https://doi.org/10.1016/j.procbio.2017.07.015.
(48) Sinirlioglu, Z. A.; Sinirlioglu, D.; Akbas, F. Preparation and Characterization of Stable Cross-Linked Enzyme Aggregates of Novel Laccase Enzyme from Shewanella putrefaciens and Using Malachite Green Decolorization. Bioresource Technology 2013, 146, 807-811. https://doi.org/10.1016/j.biortech.2013.08.032.
(49) Kim, M. H.; Park, S.; Kim, Y. H.; Won, K.; Lee, S. H. Immobilization of Formate Dehydrogenase from Candida Boidinii through Cross-Linked Enzyme Aggregates. Journal of Molecular Catalysis B: Enzymatic 2013, 97, 209-214. https://doi.org/10.1016/j.molcatb.2013.08.020.
(50) Touahar, I. E.; Haroune, L.; Ba, S.; Bellenger, J.-P.; Cabana, H. Characterization of Combined Cross-Linked Enzyme Aggregates from Laccase, Versatile Peroxidase and Glucose Oxidase, and Their Utilization for the Elimination of Pharmaceuticals. Science of The Total Environment 2014, 481, 90-99. https://doi.org/10.1016/j.scitotenv.2014.01.132.
(51) Velasco-Lozano, S.; Jackson, E.; Ripoll, M.; López-Gallego, F.; Betancor, L. Stabilization of ω-Transaminase from Pseudomonas Fluorescens by Immobilization Techniques. International Journal of Biological Macromolecules 2020, 164, 4318-4328. https://doi.org/10.1016/j.ijbiomac.2020.09.003.
(52) Khalaf, N.; Govardhan, C. P.; Lalonde, J. J.; Persichetti, R. A.; Wang, Y.-F.; Margolin, A. L. Cross-Linked Enzyme Crystals as Highly Active Catalysts in Organic Solvents. J. Am. Chem. Soc. 1996, 118 (23), 5494-5495. https://doi.org/10.1021/ja960081 s.
(53) Abdul Wahab, M. K. H.; El-Enshasy, H. A.; Bakar, F. D. A.; Murad, A. M. A.; Jahim, J. M.; Illias, R. M. Improvement of Cross-Linking and Stability on Cross-Linked Enzyme Aggregate (CLEA)-Xylanase by Protein Surface Engineering. Process Biochemistry 2019, 86, 40-49. https://doi.org/10.1016/j.procbio.2019.07.017.
(54) Abian, O.; Grazú, V.; Hermoso, J.; González, R.; García, J. L.; Fernández-Lafuente, R.; Guisán, J. M. Stabilization of Penicillin G Acylase from Escherichia Coli: Site-Directed Mutagenesis of the Protein Surface To Increase Multipoint Covalent Attachment. Applied and Environmental Microbiology 2004, 70 (2), 1249-1251. https://doi.org/10.1128/AEM.70.2.1249-1251.2004.
(55) Wright, P. E.; Dyson, H. J. Intrinsically Disordered Proteins in Cellular Signalling and Regulation. Nat Rev Mol Cell Biol 2015, 16 (1), 18-29. https://doi.org/10.1038/nrm3920.
(56) Uversky, V. N. Intrinsically Disordered Proteins and Their “Mysterious” (Meta) Physics. Frontiers in Physics 2019, 7.
(57) McCarty, J.; Delaney, K. T.; Danielsen, S. P. O.; Fredrickson, G. H.; Shea, J.-E. Complete Phase Diagram for Liquid-Liquid Phase Separation of Intrinsically Disordered Proteins. J. Phys. Chem. Lett. 2019, 10 (8), 1644-1652. https://doi.org/10.1021/acs.jpclett.9b00099.
(58) Kim, P. M.; Sboner, A.; Xia, Y.; Gerstein, M. The Role of Disorder in Interaction Networks: A Structural Analysis. Molecular Systems Biology 2008, 4 (1), 179. https://doi.org/10.1038/msb.2008.16.
(59) Dunker, A. K.; Cortese, M. S.; Romero, P.; Iakoucheva, L. M.; Uversky, V. N. Flexible Nets. The FEBS Journal 2005, 272 (20), 5129-5148. https://doi.org/10.1111/j.1742-4658.2005.04948.x.
(60) Hirose, T.; Ninomiya, K.; Nakagawa, S.; Yamazaki, T. A Guide to Membraneless Organelles and Their Various Roles in Gene Regulation. Nat Rev Mol Cell Biol 2022, 1-17. https://doi.org/10.1038/s41580-022-00558-8.
(61) Bondos, S. E.; Dunker, A. K.; Uversky, V. N. On the Roles of Intrinsically Disordered Proteins and Regions in Cell Communication and Signaling. Cell Commun Signal 2021, 19 (1), 88. https://doi.org/10.1186/s12964-021-00774-3.
(62) Tompa, P.; Davey, N. E.; Gibson, T. J.; Babu, M. M. A Million Peptide Motifs for the Molecular Biologist. Mol Cell 2014, 55 (2), 161-169. https://doi.org/10.1016/j.molcel.2014.05.032.
(63) Shea, J.-E.; Best, R. B.; Mittal, J. Physics-Based Computational and Theoretical Approaches to Intrinsically Disordered Proteins. Current Opinion in Structural Biology 2021, 67, 219-225. https://doi.org/10.1016/j.sbi.2020.12.012.
(64) Launay, H.; Receveur-Bréchot, V.; Carrière, F.; Gontero, B. Orchestration of Algal Metabolism by Protein Disorder. Archives of Biochemistry and Biophysics 2019, 672, 108070. https://doi.org/10.1016/j.abb.2019.108070.
(65) Piovesan, D.; Necci, M.; Escobedo, N.; Monzon, A. M.; Hatos, A.; Mic̆etić, I.; Quaglia, F.; Paladin, L.; Ramasamy, P.; Dosztányi, Z.; Vranken, W. F.; Davey, N. E.; Parisi, G.; Fuxreiter, M.; Tosatto, S. C. E. MobiDB: Intrinsically Disordered Proteins in 2021. Nucleic Acids Research 2021, 49 (D1), D361-D367. https://doi.org/10.1093/nar/gkaa1058.
(66) Mitrea, D. M.; Mittasch, M.; Gomes, B. F.; Klein, I. A.; Murcko, M. A. Modulating Biomolecular Condensates: A Novel Approach to Drug Discovery. Nat Rev Drug Discov 2022, 21 (11), 841-862. https://doi.org/10.1038/s41573-022-00505-4.
(67) Schuster, B. S.; Regy, R. M.; Dolan, E. M.; Kanchi Ranganath, A.; Jovic, N.; Khare, S. D.; Shi, Z.; Mittal, J. Biomolecular Condensates: Sequence Determinants of Phase Separation, Microstructural Organization, Enzymatic Activity, and Material Properties. J. Phys. Chem. B 2021, 125 (14), 3441-3451. https://doi.org/10.1021/acs.jpcb.0c11606.
(68) Weber, S. C. Sequence-Encoded Material Properties Dictate the Structure and Function of Nuclear Bodies. Current Opinion in Cell Biology 2017, 46, 62-71. https://doi.org/10.1016/j.ceb.2017.03.003.
(69) Banani, S. F.; Lee, H. O.; Hyman, A. A.; Rosen, M. K. Biomolecular Condensates: Organizers of Cellular Biochemistry. Nat Rev Mol Cell Biol 2017, 18 (5), 285-298. https://doi.org/10.1038/nrm.2017.7.
(70) Williams, D. S.; Koga, S.; Hak, C. R. C.; Majrekar, A.; Patil, A. J.; Perriman, A. W.; Mann, S. Polymer/Nucleotide Droplets as Bio-Inspired Functional Micro-Compartments. Soft Matter 2012, 8 (22), 6004-6014. https://doi.org/10.1039/C2SM25184A.
(71) Woodruff, J. B.; Ferreira Gomes, B.; Widlund, P. O.; Mahamid, J.; Honigmann, A.; Hyman, A. A. The Centrosome Is a Selective Condensate That Nucleates Microtubules by Concentrating Tubulin. Cell 2017, 169 (6), 1066-1077.e10. https://doi.org/10.1016/j.cell.2017.05.028.
(72) Solis-Miranda, J.; Chodasiewicz, M.; Skirycz, A.; Fernie, A. R.; Moschou, P. N.; Bozhkov, P. V.; Gutierrez-Beltran, E. Stress-Related Biomolecular Condensates in Plants. The Plant Cell 2023, 35 (9), 3187-3204. https://doi.org/10.1093/plcell/koad127.
(73) Lafontaine, D. L. J.; Riback, J. A.; Bascetin, R.; Brangwynne, C. P. The Nucleolus as a Multiphase Liquid Condensate. Nat Rev Mol Cell Biol 2021, 22 (3), 165-182. https://doi.org/10.1038/s41580-020-0272-6.
(74) Londoño Vélez, V.; Alquraish, F.; Tarbiyyah, I.; Rafique, F.; Mao, D.; Chodasiewicz, M. Landscape of Biomolecular Condensates in Heat Stress Responses. Frontiers in Plant Science 2022, 13.
(75) Romero-Perez, P. S.; Dorone, Y.; Flores, E.; Sukenik, S.; Boeynaems, S. When Phased without Water: Biophysics of Cellular Desiccation, from Biomolecules to Condensates. Chem. Rev. 2023, 123 (14), 9010-9035. https://doi.org/10.1021/acs.chemrev.2c00659.
(76) Huelgas-Morales, G.; Silva-Garcia, C. G.; Salinas, L. S.; Greenstein, D.; Navarro, R. E. The Stress Granule RNA-Binding Protein TIAR-1 Protects Female Germ Cells from Heat Shock in Caenorhabditis Elegans. G3 Genes|Genomes|Genetics 2016, 6 (4), 1031-1047. https://doi.org/10.1534/g3.115.026815.
(77) Wang, L.; Yang, W.; Li, B.; Yuan, S.; Wang, F. Response to Stress in Biological Disorders: Implications of Stress Granule Assembly and Function. Cell Proliferation 2021, 54 (8), e13086. https://doi.org/10.1111/cpr.13086.
(78) Soding, J.; Zwicker, D.; Sohrabi-Jahromi, S.; Boehning, M.; Kirschbaum, J. Mechanisms for Active Regulation of Biomolecular Condensates. Trends in Cell Biology 2020, 30 (1), 4-14. https://doi.org/10.1016/j.tcb.2019.10.006.
(79) Ditlev, J. A.; Case, L. B.; Rosen, M. K. Who's In and Who's Out-Compositional Control of Biomolecular Condensates. Journal of Molecular Biology 2018, 430 (23), 4666-4684. https://doi.org/10.1016/j.jmb.2018.08.003.
(80) Roden, C.; Gladfelter, A. S. RNA Contributions to the Form and Function of Biomolecular Condensates. Nat Rev Mol Cell Biol 2021, 22 (3), 183-195. https://doi.org/10.1038/s41580-020-0264-6.
(81) Elbaum-Garfinkle, S.; Kim, Y.; Szczepaniak, K.; Chen, C. C.-H.; Eckmann, C. R.; Myong, S.; Brangwynne, C. P. The Disordered P Granule Protein LAF-1 Drives Phase Separation into Droplets with Tunable Viscosity and Dynamics. Proceedings of the National Academy of Sciences 2015, 112 (23), 7189-7194. https://doi.org/10.1073/pnas.1504822112.
(82) Roberts, S.; Dzuricky, M.; Chilkoti, A. Elastin-like Polypeptides as Models of Intrinsically Disordered Proteins. FEBS Letters 2015, 589 (19, Part A), 2477-2486. https://doi.org/10.1016/j.febslet.2015.08.029.
(83) Li, L.; Tong, Z.; Jia, X.; Kiick, K. L. Resilin-like Polypeptide Hydrogels Engineered for Versatile Biological Function. Soft Matter 2012, 9 (3), 665-673. https://doi.org/10.1039/C2SM26812D.
(84) Meyer, D. E.; Chilkoti, A. Quantification of the Effects of Chain Length and Concentration on the Thermal Behavior of Elastin-like Polypeptides. Biomacromolecules 2004, 5 (3), 846-851. https://doi.org/10.1021/bm034215n.
(85) Schuster, B. S.; Dignon, G. L.; Tang, W. S.; Kelley, F. M.; Ranganath, A. K.; Jahnke, C. N.; Simpkins, A. G.; Regy, R. M.; Hammer, D. A.; Good, M. C.; Mittal, J. Identifying Sequence Perturbations to an Intrinsically Disordered Protein That Determine Its Phase-Separation Behavior. Proceedings of the National Academy of Sciences 2020, 117 (21), 11421-11431. https://doi.org/10.1073/pnas.2000223117.
(86) Klass, S. H.; Smith, M. J.; Fiala, T. A.; Lee, J. P.; Omole, A. O.; Han, B.-G.; Downing, K. H.; Kumar, S.; Francis, M. B. Self-Assembling Micelles Based on an Intrinsically Disordered Protein Domain. J. Am. Chem. Soc. 2019, 141 (10), 4291-4299. https://doi.org/10.1021/jacs.8b10688.
(87) Acosta, S.; Poocza, L.; Quintanilla-Sierra, L.; Rodriguez-Cabello, J. C. Charge Density as a Molecular Modulator of Nanostructuration in Intrinsically Disordered Protein Polymers. Biomacromolecules 2021, 22 (1), 158-170. https://doi.org/10.1021/acs.biomac.0c00934.
(88) Dai, Y.; You, L.; Chilkoti, A. Engineering Synthetic Biomolecular Condensates. Nat Rev Bioeng 2023, 1 (7), 466-480. https://doi.org/10.1038/s44222-023-00052-6.
(89) Garabedian, M. V.; Wang, W.; Dabdoub, J. B.; Tong, M.; Caldwell, R. M.; Benman, W.; Schuster, B. S.; Deiters, A.; Good, M. C. Designer Membraneless Organelles Sequester Native Factors for Control of Cell Behavior. Nat Chem Biol 2021, 17 (9), 998-1007. https://doi.org/10.1038/s41589-021-00840-4.
(90) Fletcher, E. E.; Yan, D.; Kosiba, A. A.; Zhou, Y.; Shi, H. Biotechnological Applications of Elastin-like Polypeptides and the Inverse Transition Cycle in the Pharmaceutical Industry. Protein Expression and Purification 2019, 153, 114-120. https://doi.org/10.1016/j.pep.2018.09.006.
(91) Fernandez-Lorente, G.; Rocha-Martin, J.; Guisan, J. M. Immobilization of Lipases by Adsorption on Hydrophobic Supports: Modulation of Enzyme Properties in Biotransformations in Anhydrous Media. In Immobilization of Enzymes and Cells: Methods and Protocols; Guisan, J. M., Bolivar, J. M., López-Gallego, F., Rocha-Martin, J., Eds.; Methods in Molecular Biology; Springer US: New York, NY, 2020; pp 143-158. https://doi.org/10.1007/978-1-0716-0215-7_9.
(92) Bai, H.; Xu, F.; Anjia, L.; Matsui, H. Low Temperature Synthesis of ZnO Nanowires by Using a Genetically-Modified Collagen-like Triple Helix as a Catalytic Template. Soft Matter 2009, 5 (5), 966-969. https://doi.org/10.1039/B816449E.
(93) Schacht, K.; Scheibel, T. Processing of Recombinant Spider Silk Proteins into Tailor-Made Materials for Biomaterials Applications. Current Opinion in Biotechnology 2014, 29, 62-69. https://doi.org/10.1016/j.copbio.2014.02.015.
(94) Asai, D.; Xu, D.; Liu, W.; Garcia Quiroz, F.; Callahan, D. J.; Zalutsky, M. R.; Craig, S. L.; Chilkoti, A. Protein Polymer Hydrogels by in Situ, Rapid and Reversible Self-Gelation. Biomaterials 2012, 33 (21), 5451-5458. https://doi.org/10.1016/j.biomaterials.2012.03.083.
(95) Cao, L.; van Rantwijk, F.; Sheldon, R. A. Cross-Linked Enzyme Aggregates: A Simple and Effective Method for the Immobilization of Penicillin Acylase. Org. Lett. 2000, 2 (10), 1361-1364. https://doi.org/10.1021/01005593x.
(96) Schuster, B. S.; Reed, E. H.; Parthasarathy, R.; Jahnke, C. N.; Caldwell, R. M.; Bermudez, J. G.; Ramage, H.; Good, M. C.; Hammer, D. A. Controllable Protein Phase Separation and Modular Recruitment to Form Responsive Membraneless Organelles. Nat Commun 2018, 9 (1), 2985. https://doi.org/10.1038/s41467-018-05403-1.
(97) Nadar, S. S.; Muley, A. B.; Ladole, M. R.; Joshi, P. U. Macromolecular Cross-Linked Enzyme Aggregates (M-CLEAs) of α-Amylase. International Journal of Biological Macromolecules 2016, 84, 69-78. https://doi.org/10.1016/j.ijbiomac.2015.11.082.
(98) Guo, F.; Berglund, P. Transaminase Biocatalysis: Optimization and Application. Green Chemistry 2017, 19 (2), 333-360. https://doi.org/10.1039/C6GC02328B.
(99) Desai, A. A. Sitagliptin Manufacture: A Compelling Tale of Green Chemistry, Process Intensification, and Industrial Asymmetric Catalysis. Angewandte Chemie International Edition 2011, 50 (9), 1974-1976. https://doi.org/10.1002/anie.201007051.
(100) Frodsham, L.; Golden, M.; Hard, S.; Kenworthy, M. N.; Klauber, D. J.; Leslie, K.; Macleod, C.; Meadows, R. E.; Mulholland, K. R.; Reilly, J.; Squire, C.; Tomasi, S.; Watt, D.; Wells, A. S. Use of ω-Transaminase Enzyme Chemistry in the Synthesis of a JAK2 Kinase Inhibitor. Org. Process Res. Dev. 2013, 17 (9), 1123-1130. https://doi.org/10.1021/op400133d.
(101) Baud, D.; Ladkau, N.; S. Moody, T.; M. Ward, J.; C. Hailes, H. A Rapid, Sensitive Colorimetric Assay for the High-Throughput Screening of Transaminases in Liquid or Solid-Phase. Chemical Communications 2015, 51 (97), 17225-17228. https://doi.org/10.1039/C5CC06817G.
(102) Czarnievicz, N.; Rubanu, M. G.; Iturralde, M.; Albarran-Velo, J.; Diamanti, E.; Gotor-Fernandez, V.; Skolimowski, M.; López-Gallego, F. A Multiplex Assay to Assess the Transaminase Activity toward Chemically Diverse Amine Donors. ChemBioChem 2023, 24 (2), e202200614.https://doi.org/10.1002/cbic.202200614.
(103) Roura Padrosa, D.; Alaux, R.; Smith, P.; Dreveny, I.; López-Gallego, F.; Paradisi, F. Enhancing PLP-Binding Capacity of Class-III ω-Transaminase by Single Residue Substitution. Frontiers in Bioengineering and Biotechnology 2019, 7.
(104) Humble, M. S.; Cassimjee, K. E.; Håkansson, M.; Kimbung, Y. R.; Walse, B.; Abedi, V.; Federsel, H.-J.; Berglund, P.; Logan, D. T. Crystal Structures of the Chromobacterium violaceumω-Transaminase Reveal Major Structural Rearrangements upon Binding of Coenzyme PLP. The FEBS Journal 2012, 279 (5), 779-792. https://doi.org/10.1111/j.1742-4658.2012.08468.x.
(105) Meyer, D. E.; Chilkoti, A. Purification of Recombinant Proteins by Fusion with Thermally-Responsive Polypeptides. Nat Biotechnol 1999, 17 (11), 1112-1115. https://doi.org/10.1038/15100.
(106) Steffen-Munsberg, F.; Vickers, C.; Kohls, H.; Land, H.; Mallin, H.; Nobili, A.; Skalden, L.; van den Bergh, T.; Joosten, H.-J.; Berglund, P.; Höhne, M.; Bornscheuer, U. T. Bioinformatic Analysis of a PLP-Dependent Enzyme Superfamily Suitable for Biocatalytic Applications. Biotechnology Advances 2015, 33 (5), 566-604. https://doi.org/10.1016/j.biotechadv.2014.12.012.
(107) Zakeri, B.; Fierer, J. O.; Celik, E.; Chittock, E. C.; Schwarz-Linek, U.; Moy, V. T.; Howarth, M. Peptide Tag Forming a Rapid Covalent Bond to a Protein, through Engineering a Bacterial Adhesin. Proc Natl Acad Sci USA 2012, 109 (12), E690-697. https://doi.org/10.1073/pnas.1115485109.
(108) Keeble, A. H.; Turkki, P.; Stokes, S.; Khairil Anuar, I. N. A.; Rahikainen, R.; Hytönen, V. P.; Howarth, M. Approaching Infinite Affinity through Engineering of Peptide-Protein Interaction. Proc Natl Acad Sci USA 2019, 116 (52), 26523-26533. https://doi.org/10.1073/pnas.1909653116.
(109) Khairil Anuar, I. N. A.; Banerjee, A.; Keeble, A. H.; Carella, A.; Nikov, G. I.; Howarth, M. Spy&Go Purification of SpyTag-Proteins Using Pseudo-SpyCatcher to Access an Oligomerization Toolbox. Nat Commun 2019, 10 (1), 1734. https://doi.org/10.1038/s41467-019-09678-w.
(110) Gao, X.; Fang, J.; Xue, B.; Fu, L.; Li, H. Engineering Protein Hydrogels Using SpyCatcher-SpyTag Chemistry. Biomacromolecules 2016, 17 (9), 2812-2819. https://doi.org/10.1021/acs.biomac.6b00566.
(111) Zhang, W.-B.; Sun, F.; Tirrell, D. A.; Arnold, F. H. Controlling Macromolecular Topology with Genetically Encoded SpyTag-SpyCatcher Chemistry. J. Am. Chem. Soc. 2013, 135 (37), 13988-13997. https://doi.org/10.1021/ja4076452.
(112) Zhang, G.; Johnston, T.; Quin, M. B.; Schmidt-Dannert, C. Developing a Protein Scaffolding System for Rapid Enzyme Immobilization and Optimization of Enzyme Functions for Biocatalysis. ACS Synth. Biol. 2019, 8 (8), 1867-1876. https://doi.org/10.1021/acssynbio.9b00187.
(113) Li, Y.; L. Ogorzalek, T.; Wei, S.; Zhang, X.; Yang, P.; Jasensky, J.; L. Brooks, C.; G. Marsh, E. N.; Chen, Z. Effect of Immobilization Site on the Orientation and Activity of Surface-Tethered Enzymes. Physical Chemistry Chemical Physics 2018, 20 (2), 1021-1029. https://doi.org/10.1039/C7CP06063G.
(114) Rusin, K. M.; Fare, T. L.; Stemple, J. Z. Immobilization of Flavoproteins on Silicon: Effect of Cross-Linker Chain Length on Enzyme Activity. Biosensors and Bioelectronics 1992, 7 (5), 367-373. https://doi.org/10.1016/0956-5663(92)85033-7.
(115) Rozbeský, D.; Rosůlek, M.; Kukačka, Z.; Chmelík, J.; Man, P.; Novák, P. Impact of Chemical Cross-Linking on Protein Structure and Function. Anal. Chem. 2018, 90 (2), 1104-1113. https://doi.org/10.1021/acs.analchem.7b02863.
(116) Velasco-Lozano, S.; López-Gallego, F.; Vázquez-Duhalt, R.; Mateos-Díaz, J. C.; Guisán, J. M.; Favela-Torres, E. Carrier-Free Immobilization of Lipase from Candida rugosa with Polyethyleneimines by Carboxyl-Activated Cross-Linking. Biomacromolecules 2014, 15 (5), 1896-1903. https://doi.org/10.1021/bm500333v.
(117) Keeble, A. H.; Yadav, V. K.; Ferla, M. P.; Bauer, C. C.; Chuntharpursat-Bon, E.; Huang, J.; Bon, R. S.; Howarth, M. DogCatcher Allows Loop-Friendly Protein-Protein Ligation. Cell Chemical Biology 2022, 29 (2), 339-350.e10. https://doi.org/10.1016/j.chembiol.2021.07.005.
(118) Sperl, J. M.; Sieber, V. Multienzyme Cascade Reactions—Status and Recent Advances. ACS Catal. 2018, 8 (3), 2385-2396. https://doi.org/10.1021/acscatal.7b03440.
(119) Köhler, V.; J. Turner, N. Artificial Concurrent Catalytic Processes Involving Enzymes. Chemical Communications 2015, 51 (3), 450-464. https://doi.org/10.1039/C4CC07277D.
(120) Arana-Peña, S.; Carballares, D.; Morellon-Sterlling, R.; Berenguer-Murcia, Á.; Alcántara, A. R.; Rodrigues, R. C.; Fernandez-Lafuente, R. Enzyme Co-Immobilization: Always the Biocatalyst Designers' Choice . . . or Not? Biotechnology Advances 2021, 51, 107584. https://doi.org/10.1016/j.biotechadv.2020.107584.
(121) Perwez, M.; Ahmed Mazumder, J.; Sardar, M. Preparation and Characterization of Reusable Magnetic Combi-CLEA of Cellulase and Hemicellulase. Enzyme and Microbial Technology 2019, 131, 109389. https://doi.org/10.1016/j.enzmictec.2019.109389.
(122) Peng, F.; Chen, Q.-S.; Zong, M.-H.; Lou, W.-Y. Sequential Co-Immobilization of Multienzyme Nanodevices Based on SpyCatcher and SpyTag for Robust Biocatalysis. Molecular Catalysis 2021, 510, 111673. https://doi.org/10.1016/j.mcat.2021.111673.
(123) Zhang, G.; Quin, M. B.; Schmidt-Dannert, C. Self-Assembling Protein Scaffold System for Easy in Vitro Coimmobilization of Biocatalytic Cascade Enzymes. ACS Catal. 2018, 8 (6), 5611-5620.https://doi.org/10.1021/acscatal.8b00986.
(124) Heinks, T.; Koopmeiners, S.; Montua, N.; Sewald, N.; Höhne, M.; Bornscheuer, U. T.; Fischer von Mollard, G. Co-Immobilization of a Multi-Enzyme Cascade: (S)-Selective Amine Transaminases, 1-Amino Acid Oxidase and Catalase. ChemBioChem n/a (n/a), e202300425. https://doi.org/10.1002/cbic.202300425.
(125) Williams, V.; Cui, Y.; Jiang, X.; Zhang, C.; Zhao, J.; Zhang, N. Co-Immobilized Multienzyme System for the Cofactor-Driven Cascade Synthesis of (R)-2-Amino-3-(2-Bromophenyl) Propanoic Acid: A Model Reaction. Org. Process Res. Dev. 2022, 26 (11), 3024-3033. https://doi.org/10.1021/acs.oprd.2c00119.
(126) Kelley, F. M.; Favetta, B.; Regy, R. M.; Mittal, J.; Schuster, B. S. Amphiphilic Proteins Coassemble into Multiphasic Condensates and Act as Biomolecular Surfactants. Proceedings of the National Academy of Sciences 2021, 118 (51), e2109967118. https://doi.org/10.1073/pnas.2109967118.
(127) Velasco-Lozano, S.; Santiago-Arcos, J.; Mayoral, J. A.; López-Gallego, F. Co-Immobilization and Colocalization of Multi-Enzyme Systems for the Cell-Free Biosynthesis of Aminoalcohols. ChemCatChem 2020, 12 (11), 3030-3041. https://doi.org/10.1002/cctc.201902404.
(128) Meyer, D/and Chilkoti, S. “18: Protein purification by inverse transition cycling.” Protein-protein interactions: A molecular cloning manual (2002): 329-344
(129) Li, X., Kuchinski, L. M., Park, A., Murphy, G. S., Soto, K. C., & Schuster, B. S. (2024). Enzyme purification and sustained enzyme activity for pharmaceutical biocatalysis by fusion with phase-separating intrinsically disordered protein. Biotechnology and Bioengineering, 1-14.
(130) Schuster, B. S., Dignon, G. L., Tang, W., Kelley, F. M., Ranganath, A. K., Jahnke, C. N., . . . & Mittal, J. (2020). Identifying sequence perturbations to an intrinsically disordered protein that determine its phase-separation behavior. Proceedings of the National Academy of Sciences, 117(21), 11421-11431.

### EMBODIMENTS

1. A biomolecular condensate comprising a plurality of crosslinked, immobilized fusion proteins; said fusion proteins having at least one first protein of interest operably linked to at least one heterologous intrinsically disordered region (IDR) sequence; said crosslinked biomolecular condensate lacking liquid-like properties.
2. The biomolecular condensate of embodiment 1, wherein the first protein of interest is an enzyme which retains catalytic activity after crosslinking.
3. The biomolecular condensate of any one of embodiments 1 or 2, wherein the first protein of interest has at least one reactive surface residue.
4. The biomolecular condensate of any one of the preceding embodiments, wherein the first protein of interest is an alcohol dehydrogenase, ketoreductase, alcohol oxidase, transaminase, monooxygenase, acylase, lipase, esterase, thioesterase, caboxylic acid reductase, aldolase, amine dehydrogenase, polymerase, lactase, or amylase.
5. The biomolecular condensate of any one of the preceding embodiments, wherein the first protein of interest is a ω-transaminase.
6. The biomolecular condensate of any one of embodiment 2 to embodiment 5 wherein said particle comprises a second, third or fourth protein of interest operably linked to an IDR; said second, third or fourth protein of interest being different enzymes from said first protein of interest and retaining catalytic activity; said multiple enzymes forming a coordinated biocatalytic cascade that efficiently catalyze multistep enzymatic reactions.
7. The biomolecular condensate of any one of the preceding embodiments, wherein the IDR drives phase separation.
8. The biomolecular condensate of any one of the preceding embodiments, wherein the IDR is RGG or PGL3.
9. The biomolecular condensate of embodiment 8, wherein the IDR is RGG.
10. The biomolecular condensate of any one of the preceding embodiments, wherein the fusion proteins are cross-linked by the formation of an isopeptide bond or a disulfide bond.
11. The biomolecular condensate of any one of the preceding embodiments, wherein the fusion proteins are cross-linked using glutaraldehyde, BS3, bis-maleimide, or other crosslinking reagents with reactivity towards amines or sulfhydryls.
12. The biomolecular condensate of any one of embodiments 1-11, wherein the plurality of fusion proteins present in the particle are not identical.
13. The biomolecular condensate of any one of embodiments 1-12, wherein the biomolecular condensate has a diameter of between about 0.25 μm-about 20 μm.
14. The biomolecular condensate of embodiment 13, wherein the biomolecular condensate has a diameter of between about between 0.25 μm-4 μm.
15. A solid support comprising the biomolecular condensate of any one of embodiments 1-14.
16. A method for producing an immobile, biologically active biomolecular condensate particle comprising at least one IDR operably linked to a first enzyme, comprising:
   a. providing a cell comprising a fusion protein having at least one protein of interest and at least one intrinsically disordered region (IDR) sequence;
   b. lysing said cell thereby forming a lysate;
   c. collecting the fusion protein from said lysate using liquid-liquid phase separation (LLPS) and/or chromatography,
   d. contacting the fusion protein of step c) with a crosslinking agent under conditions suitable for crosslinking to occur while maintaining catalytic activity of said enzyme such that a particle is formed containing crosslinked fusion protein; and, optionally
   e. isolating said plurality of crosslinked IDR-catalytically active enzyme fusion protein containing particles.
17. The method of embodiment 16 wherein said fusion protein comprises and at least one IDR and an enzyme selected from an alcohol dehydrogenase, ketoreductase, alcohol oxidase, transaminase, monooxygenase, acylase, lipase, esterase, thioesterase, caboxylic acid reductase, aldolase, amine dehydrogenase, polymerase, lactase, or amylase.
18. The method of embodiment 16 or 17, wherein the conditions comprise modulating at least one parameter selected from cross-linking agent concentrations, cross-linking temperature, and time period for cross linking to occur.
19. The method of embodiment 18, wherein the conditions comprise 0-4° C. for 1 hour.
20. The method of any one of embodiments 16-19, wherein said cross linking agent is selected from the group consisting of glutaraldehyde, BS3, bis-maleimide, or other crosslinking reagents with reactivity towards amines or sulfhydryls.
21. The method of anyone of embodiments 16-20, wherein the fusion proteins are cross-linked by the formation of an isopeptide bond.
22. The method of anyone of embodiments 16-21, wherein the at least two fusion proteins comprise catalytically active enzymes that are not identical, and form a coordinated multi-enzyme biocatalytic cascade that efficiently catalyzes multistep enzymatic reactions.
23. The method of any one of embodiments 16-22, wherein crosslinking conditions generate a crosslinked biomolecular condensate with a diameter between 0.25 μm-20 μm.
24. The method of embodiment 23, wherein the diameter is between 0.25 μm-4 μm.
25. The method of any one of embodiments 16-24, wherein the chromatography is Ni-NTA affinity chromatography.
26. The method of any one of embodiments 16-25, wherein the cells are lysed by sonication.
27. The method of any one of embodiments 16-26, wherein the crosslinking reaction is quenched prior to isolating the fusion protein.
28. The method of embodiment 27, wherein the cross-linking reaction is quenched by addition of tris(hydroxymethyl)aminomethane (Tris).
29. The method of any one of embodiments 16-28, further comprising centrifuging the lysate prior to step d).
30. The method of any one of embodiments 16-29, further comprising detecting the formation of the crosslinked fusion proteins.
31. The method of any one of embodiments 16-30, wherein the crosslinked, enzymatically active biomolecular condensate does not exhibit liquid-like properties.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A biomolecular condensate particle comprising a plurality of crosslinked, immobilized fusion proteins; said fusion proteins having at least one first protein of interest operably linked to at least one heterologous intrinsically disordered region (IDR) sequence; said crosslinked biomolecular condensate lacking liquid-like properties.

2. The biomolecular condensate of claim 1, wherein
a. the first protein of interest is an enzyme which retains catalytic activity after crosslinking;
b. the first protein of interest has at least one reactive surface residue;
c. the first protein of interest is an alcohol dehydrogenase, ketoreductase, alcohol oxidase, transaminase, monooxygenase, acylase, lipase, esterase, thioesterase, caboxylic acid reductase, aldolase, amine dehydrogenase, polymerase, lactase, or amylase; or
d. the first protein of interest is a ω-transaminase.

3. The biomolecular condensate of claim 1, wherein said particle comprises a second, third or fourth protein of interest operably linked to an IDR; said second, third or fourth protein of interest being different enzymes from said first protein of interest and retaining catalytic activity; said multiple enzymes forming a coordinated biocatalytic cascade that efficiently catalyze multistep enzymatic reactions.

4. The biomolecular condensate of claim 1, wherein
a. the IDR drives phase separation; or
b. the IDR is RGG or PGL3.

5. The biomolecular condensate of claim 4, wherein the IDR is RGG.

6. The biomolecular condensate of claim 1, wherein
a. the fusion proteins are cross-linked by the formation of an isopeptide bond or a disulfide bond; or
b. the fusion proteins are cross-linked using glutaraldehyde, BS3, bis-maleimide, or other crosslinking reagents with reactivity towards amines or sulfhydryls.

7. The biomolecular condensate of claim 1, wherein the plurality of fusion proteins present in the particle are not identical.

8. The biomolecular condensate of claim 1, wherein the biomolecular condensate has a diameter of between about 0.25 μm-about 20 μm, or between about 0.25 μm-4 μm.

9. A solid support comprising the biomolecular condensate of claim 1.

10. A method for producing an immobile, biologically active biomolecular condensate particle comprising at least one IDR operably linked to a first enzyme, comprising:
a. providing a cell comprising a fusion protein having at least one protein of interest and at least one intrinsically disordered region (IDR) sequence;
b. lysing said cell thereby forming a lysate;
c. collecting the fusion protein from said lysate,
d. contacting the fusion protein of step c) with a crosslinking agent under conditions suitable for crosslinking to occur while maintaining catalytic activity of said enzyme such that a particle is formed containing crosslinked fusion protein; and, optionally
e. isolating said plurality of crosslinked IDR-catalytically active enzyme fusion protein containing particles.

11. The method of claim 10 wherein said fusion protein comprises and at least one IDR and an enzyme selected from an alcohol dehydrogenase, ketoreductase, alcohol oxidase, transaminase, monooxygenase, acylase, lipase, esterase, thioesterase, caboxylic acid reductase, aldolase, amine dehydrogenase, polymerase, lactase, or amylase.

12. The method of claim 10, wherein the conditions comprise modulating at least one parameter selected from cross-linking agent concentrations, cross-linking temperature, and time period for cross linking to occur.

13. The method of claim 12, wherein the conditions comprise 0-4° C. for 1 hour.

14. The method of claim 10, wherein said cross linking agent is selected from the group consisting of glutaraldehyde, BS3, bis-maleimide, or other crosslinking reagents with reactivity towards amines or sulfhydryls.

15. The method of claim 10, wherein
a. the fusion proteins are recovered using chromatography and are cross-linked by formation of an isopeptide bond; or
b. at least two fusion proteins are present, comprise catalytically active enzymes that are not identical, and form a coordinated multi-enzyme biocatalytic cascade that efficiently catalyzes multistep enzymatic reactions.

16. The method of claim 10, wherein crosslinking conditions generate a crosslinked biomolecular condensate with a diameter between 0.25 μm-20 μm or between 0.25 μm-4 μm.

17. The method of claim 10, wherein fusion protein is recovered using liquid-liquid phase separation (LLPS) and/or Ni-NTA affinity chromatography.

18. The method of claim 10, wherein the crosslinking reaction is quenched prior to isolating the fusion protein, optionally by addition of tris(hydroxymethyl)aminomethane (Tris).

19. The method of claim 10, further comprising
a. centrifuging the lysate prior to step d); or
b. detecting the formation of the crosslinked fusion proteins.

20. The method of claim 10, wherein the crosslinked, enzymatically active biomolecular condensate does not exhibit liquid-like properties.
